(19) United States
(12) Patent Application Publication
(10) Pub. No.: US 2010/0016435 A1
(43) Pub. Date: Jan. 21, 2010

(54) HYBRID MOLECULES HAVING MIXED VITAMIN D RECEPTOR AGONISM AND HISTONE DEACETYLASE INHIBITORY PROPERTIES

(75) Inventors: John White, Town of Mount Royal (CA); Jim Gleason, MOntreal (CA); Luz Elisa Tavera Mendoza, Longueuil (CA); Tan Quach, Scarborough (CA)

Correspondence Address: FULBRIGHT & JAWORSKI L.L.P., 600 CONGRESS AVE., SUITE 2400, AUSTIN, TX, 78701, US

(73) Assignee: McGill University, Montreal, QC (CA)

(21) Appl. No.: 12/300,984

(22) PCT Filed: May 16, 2007
(86) PCT No.: PCT/CA07/00885

§ 371 Date: Jun. 3, 2009

Related U.S. Application Data
(60) Provisional application No. 60/800,424, filed on May 16, 2006.

(51) Int. Cl. A61K 31/16; A61K 31/19; A61P 35/00

(52) U.S. Cl. 514/563; 514/557

(57) ABSTRACT

Novel chemical agents are described. More specifically, hybrid molecules comprising a vitamin D receptor agonist moiety and an HDAC inhibitor moiety are described herein.

### CROSS REFERENCE TO RELATED APPLICATIONS

The present application claims the benefit of U.S. Provisional Application No. 60/800,424 filed May 16, 2006, the entire contents of which are incorporated by reference.

### FIELD OF THE INVENTION

The present invention relates to a series of new chemical agents that demonstrate antiproliferative and cytotoxic activity against cancer cells. More particularly, but not exclusively, the present invention relates to hybrid molecules capable of mixed vitamin D receptor agonism and histone deacetylase inhibition. The present invention also relates to methods of their synthesis.

### BACKGROUND OF THE INVENTION

1α,25-Dihydroxyvitamin D₃ (Calcitriol, 1), the biologically active metabolite of vitamin D₃ (Calciferol, 2), is a primary physiological regulator of calcium homeostasis, controlling intestinal calcium absorption, bone resorption and bone mineralization.¹⁻³

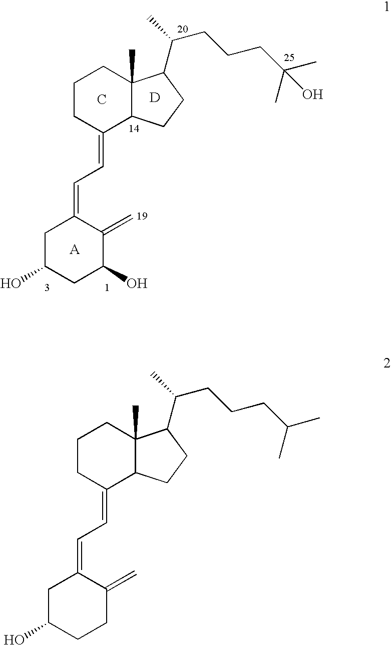

The vitamin D receptor (VDR), a member of the nuclear receptor family of ligand-regulated transcription factors, plays a crucial role in calcitriol's signaling. Calcitriol-bound VDR heterodimerizes with related retinoid X receptors and binds to specific DNA sequences called vitamin D response elements, located in the regulatory regions of target genes.²

Calcitriol has been reported as regulating cell differentiation and cell proliferation, as well as having anti-cancer properties.^{2,3} However, the calcemic activity of calcitriol has limited its use in the treatment of cancers due to hypercalcemia typically induced by the required supraphysiological levels of the compound in these treatments.

Intensive efforts have been devoted to the development of calcitriol analogues (e.g. suberoylanilide hydroxamic acid, SAHA, 4) that would combine therapeutic potential with lowered calcemic activity.⁴⁻⁶ Structural changes and modifications in strategic locations throughout the calcitriol molecular backbone have led to the development of numerous analogues, many of which have shown potent antiproliferative and antidifferentiation activities, along with desired lowered calcemic activity.⁴ Several of these analogues have advanced to preclinical studies for the treatment of diverse human diseases, and some have become FDA-approved drugs.⁷

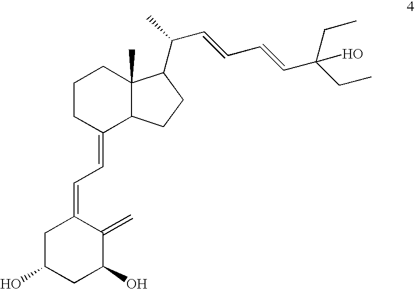

Cancer progression has been associated with an acquired resistance to calcitriol. This resistance however, can be overcome by co-administration of the histone deacetylase (HDAC) inhibitor trichostatin A (TSA, 3). HDAC inhibitors (a further example of which is provided by 5) have emerged as a new and promising class of anticancer agents, capable of regulating transcription and inhibiting cancer cell proliferation by induction of cell cycle arrest in either G0/G1 or G2/M, cell differentiation and/or apoptosis.^{8,9}

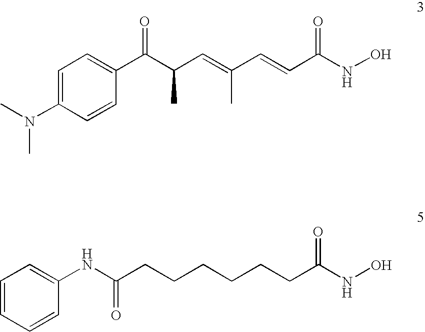

The present description refers to a number of documents, the content of which is herein incorporated by reference in their entirety.

### SUMMARY OF THE INVENTION

The present invention relates to hybrid molecules capable of mixed vitamin D receptor agonism and histone deacetylase inhibition.

In an embodiment, the present invention relates to hybrid molecules comprising a vitamin D receptor agonist moiety and an HDAC inhibitor moiety.

In an embodiment, the present invention relates to hybrid molecules comprising a vitamin D receptor agonist moiety and an HDAC inhibitor moiety, wherein the HDAC inhibitor moiety is modelled after an HDAC inhibitor selected from the group consisting of TSA, sodium butyrate (NaB), valproic acid, N-acetyldinaline, and suberoylanilide hydroxamic acid (SAHA).

In an embodiment, the present invention relates to hybrid molecules or pharmaceutically acceptable salts thereof selected from the group consisting of:

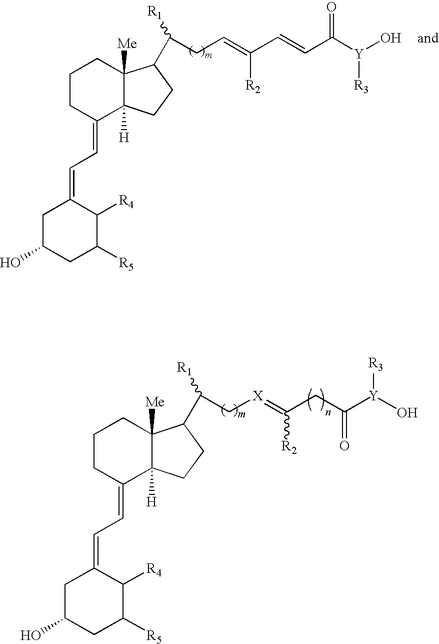

wherein:

R₁, R₂, R₃, and R₄ are independently selected from the group consisting of H, lower alkyl, and alkylene;

R₅ is selected from the group consisting of H and OH;

X is selected from the group consisting of O, S NH and CH₂;

Y is selected from the group consisting of N and CH;

m is an integer ranging from 0 to 3; and

n is an integer ranging from 1 to 3.

In an embodiment, the present invention relates to a hybrid molecule, or a pharmaceutically acceptable salt or prodrug thereof, comprising the structure:

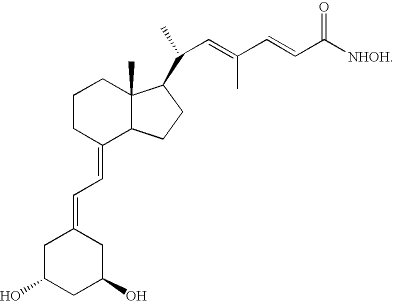

In an embodiment, the present invention relates to a hybrid molecule, or a pharmaceutically acceptable salt or prodrug thereof, comprising the structure:

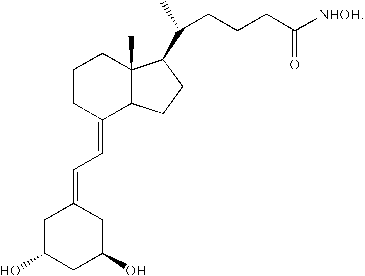

In an embodiment, the present invention relates to a hybrid molecule, or a pharmaceutically acceptable salt or prodrug thereof, comprising the structure:

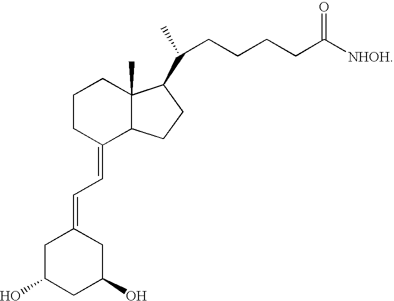

In an embodiment, the present invention relates to a method for the treatment of disorders or diseases wherein inhibition of HDAC and/or vitamin D agonism is beneficial, the method comprising administering to a subject in need thereof and affective amount of one or more hybrid molecules as disclosed herein.

In an embodiment, the present invention relates to a method of treating a patient afflicted with a condition selected from the group consisting of cancer, inflammation and auto-immune diseases, comprising administering to the patient a therapeutically effective amount of one or more of the hybrid molecules as disclosed herein.

In an embodiment, the present invention relates to a method of wound healing comprising, administering to a patient in need thereof a therapeutically effective amount of one or more of the hybrid molecules as disclosed herein.

In an embodiment, the present invention relates to a method of treating bacterial infections in a patient comprising, administering to the patient a therapeutically effective amount of one or more of the hybrid molecules as disclosed herein.

In an embodiment, the present invention relates to a method of reducing proliferation of/or inducing cell death in neoplastic cells comprising, contacting the neoplastic cells with one or more of the hybrid molecules as disclosed herein.

In an embodiment, the present invention relates to a use of one or more of the hybrid molecules as disclosed herein in the manufacture of a medicament for the treatment of a condition selected from the group consisting of cancer, inflammation and auto-immune diseases.

In an embodiment, the present invention relates to a use of one or more of the hybrid molecules as disclosed herein in the manufacture of a medicament for inducing wound healing.

In an embodiment, the present invention relates to a use of one or more of the hybrid molecules as disclosed herein in the manufacture of a medicament for treating bacterial infections.

In an embodiment, the present invention relates to a pharmaceutical composition comprising an effective amount of one or more of the hybrid molecules as disclosed herein in association with one or more pharmaceutically acceptable carriers, excipients or diluents.

In an embodiment, the present invention relates to an admixture comprising an effective amount of one or more of the hybrid molecules as disclosed herein in association with one or more pharmaceutically acceptable carriers, excipients or diluents.

The foregoing and other objects, advantages and features of the present invention will become more apparent upon reading of the following non restrictive description of illustrative embodiments thereof, given by way of example only with reference to the accompanying drawings.

### BRIEF DESCRIPTION OF THE DRAWINGS

In the appended drawings:

FIG. 1 is an illustration of the minimized optimal docking structure of 1 (A) and 6 (B) bound to the VDR ligand binding domain (VDR-LBD), as obtained using AutoDoc 3.0.® The receptor is shown as ribbons with the side chains of the amino acid residues and the ligands displayed as Corey-Pauling-Koltun sticks. Contact residues are labeled in white, with predicted hydrogen bonds labeled in green. The two hydroxyl moieties of the A-ring of 1 are within hydrogen bonding distance of polar residues found in the VDR-LBD, whereas the 25-OH is located between H305 and H397 (A). Twisting of the dienyl side chain of 6 relative to the position of the side chain in 1, places the hydroxamate OH within hydrogen bonding distance of H397 (B). Overlay of 1 and 6 in the VDR is shown in C.

FIG. 2 is an illustration of the vitamin D receptor agonist activity of 6 using a reporter gene assay in transiently transfected COS7 cells. Because 4 modestly enhanced expression from the internal control plasmid expressing β-galactosidase, the data are shown un-normalized with data for β-galactosidase expression as an inset.

FIG. 3 is an illustration of the VDR agonist activity of 1, 6, 36 (A) and 1 and 39 (B) measured by determining induction of expression of the gene encoding CYP24 by reverse transcription/PCR in human head and neck squamous carcinoma cell (HNSCC) line SCC4. SCC4 cells were treated with compound concentrations ranging from 0 (−) to 10⁻⁶ molar, as indicated.

FIG. 4 is an illustration of the HDAC inhibitory activity of 3 in the SCC4 cell line. FIG. 4A is an illustration of a western blot of nuclear and cytoplasmic extracts of SCC4 cells treated with vehicle (−), 1, or 3 alone or in combination, as indicated. The 55 kDa band corresponds to the molecular weight of tubulin. FIG. 4B illustrates a western blot probed with an antibody directed against acetylated histone H4 (AcH4) showing the effects of the treatments described in A on acetylation of histone H4. FIG. 4C illustrates western blots of the effects of treatments described in A on levels of total alpha-tubulin (left) and acetylated alpha-tubulin (right).

FIG. 5 is a comparison of the capability of 3 and 6 (20 nM and 200 nM) in blocking deacetylation of a substrate that absorbs at 405 nm in its deacetylated form.

FIG. 6 is a comparison of the HDAC inhibitory activities of 1, 3, 6, 36 or 39 as assessed by their effects on acetylation of alpha-tubulin. In A, SCC4 cells were incubated with vehicle or compound 1 (10⁻⁶M), 3 (15 nM), 6 (10⁻⁶M) and 36 (10⁻⁶M), as indicated for 6 h and protein extracts were probed for total α-tubulin (Tub.) or acetylated α-tubulin (AcTub.) by Western blotting. Blots for acetylated alpha-tubulin and total alpha-tubulin are shown. In B, SCC4 cells were incubated with vehicle or compound 3 (15 nM), 6 (10⁻⁶M) or 36 (10⁻⁶M) as indicated for 6 h or 24 h and protein extracts were probed for acetylated α-tubulin (AcTub.) by Western blotting. In C, SCC4 cells were incubated with vehicle or compounds 3 (15 nM), 36 (10⁻⁶ or 10⁻⁷M) or 39 (10⁻⁶ or 10⁻⁷M) as indicated for 6 h or 24 h and protein extracts were probed for total α-tubulin (tub.) or acetylated α-tubulin (AcTub.) by Western blotting.

FIG. 7 is an illustration of the antiproliferative activities of 1 and 3, individually or in combination, in SCC4 cells (A) or in SCC25 cells (B) at the concentrations indicated.

FIG. 8 is a comparative illustration of the antiproliferative activities of 1 and 6 in the SCC4 HNSCC (A) and MDA-MB231 (B) breast cancer cell lines at the concentrations indicated.

FIG. 9 is an illustration of the effects of 1, 3, 1+3, 6 and 36 administered at the concentrations indicated on cell viability in two models. The viability of MCF-7 breast cancer cells was monitored after 24 h of incubation using a trypan blue dye exclusion assay (A). The induction of annexin V, a marker of apoptotic cell death in SCC4 cells, was measured by FACS analysis (B); 6 induced substantially higher levels of annexin V staining than 3, 1 or 1+3 after pretreatment with UV light, which sensitizes cells to apoptotic cell death.

FIG. 10 is an illustration of the effects of 1, 3, 1+3 and 6 on the induction of acidic β-galactosidase activity, a marker of autophagy (A); FIG. 10 is an illustration of the effects 1, 3, 1+3 and 6 on the levels of β-galactosidase expression as obtained by measuring the indigo cleavage product of X-Gal at 620 nm (B).

FIG. 11 is an illustration of the results obtained by fluorescence-activated cell sorting (FACS) analysis on the distribution of SCC4 cells in the cell cycle following treatment with vehicle (−), 1, or 1 and 3 together at the concentrations indicated. The results show that combined treatment with 1 and 3 induces accumulation of cells in the G2/M phase of the cell cycle.

FIG. 12 is an illustration of the immunocytochemical analysis of the effects of 1, 3, or 1 and 3 combined on SCC4 cells (A). The formation of tubulin bridges, corresponding to collapsed mitotic spindles (arrows), as well as an increased number of cell divisions (asterisk), was seen only in cells treated with 1 and 3 together. Both phenotypes are characteristics of mitotic catastrophe. The lower panels (B) show cytoplasmic bridges between SCC4 cells treated with 1 and 3, consistent with the formation of the tubulin bridges seen above in A.

### DETAILED DESCRIPTION OF ILLUSTRATIVE EMBODIMENTS

In order to provide a clear and consistent understanding of the terms used in the present specification, a number of definitions are provided below. Moreover, unless defined otherwise, all technical and scientific terms as used herein have the same meaning as commonly understood to one of ordinary skill in the art to which this invention pertains.

The use of the word “a” or “an” when used in conjunction with the term “comprising” in the claims and/or the specification may mean “one”, but it is also consistent with the meaning of “one or more”, “at least one”, and “one or more than one”. Similarly, the word “another” may mean at least a second or more.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “about” is used to indicate that a value includes an inherent variation of error for the device or the method being employed to determine the value.

The present description refers to a number of chemical terms and abbreviations used by those skilled in the art. Nevertheless, definitions of selected terms are provided for clarity and consistency.

Abbreviations: NMR: Nuclear Magnetic Resonance; MS: Mass Spectrometry; m.p.: melting point; HRMS: High Resolution Mass Spectrometry; EtOAc: Ethyl Acetate; CH₂Cl₂: Dichloromethane; CDCl₃: Chloroform-d; DMAP: 4-(N,N-dimethylamino)pyridine; TFA: Trifluoroacetic acid; TCDI: 1,1-thiocarbonyldiimidazole; AcOH: Acetic acid; TLC: Thin Layer Chromatography; FAB: Fast Atom Bombardment; FCC: Flash Column Chromatography.

As used herein, the term “alkyl” can be straight-chain or branched. This also applies if they carry substituents or occur as substituents on other residues, for example in alkoxy residues, alkoxycarbonyl residues or arylalkyl residues. Substituted alkyl residues can be substituted in any suitable position. Examples of alkyl residues containing from 1 to 18 carbon atoms are methyl, ethyl, propyl, butyl, pentyl, hexyl, heptyl, octyl, nonyl, decyl, undecyl, dodecyl, tetradecyl, hexadecyl and octadecyl, the n-isomers of all these residues, isopropyl, isobutyl, isopentyl, neopentyl, isohexyl, isodecyl, 3-methylpentyl, 2,3,4-trimethylhexyl, sec-butyl, tert-butyl, or tert-pentyl. A specific group of alkyl residues is formed by the residues methyl, ethyl, n-propyl, isopropyl, n-butyl, isobutyl, sec-butyl and tert-butyl.

As used herein, the term “lower alkyl” can be straight-chain or branched. This also applies if they carry substituents or occur as substituents on other residues, for example in alkoxy residues, alkoxycarbonyl residues or arylalkyl residues. Substituted alkyl residues can be substituted in any suitable position. Examples of lower alkyl residues containing from 1 to 6 carbon atoms are methyl, ethyl, propyl, isopropyl, butyl, isobutyl, tert-butyl, pentyl, isopentyl, neopentyl, and hexyl.

As used herein, the term “alkylene” can be a linear saturated divalent hydrocarbon radical of one to six carbon atoms or a branched saturated divalent hydrocarbon radical of three to six carbon atoms. Examples of alkylene residues are methylene, ethylene, 2,2-dimethylethylene, propylene, 2-methylpropylene, butylene, and pentylene.

As used herein the term “alkenyl” can be straight-chain or branched unsaturated alkyl residues that contain one or more, for example one, two or three double bonds which can be in any suitable position. Of course, an unsaturated alkyl residue has to contain at least two carbon atoms. Examples of unsaturated alkyl residues are alkenyl residues such as vinyl, 1-propenyl, allyl, butenyl or 3-methyl-2-butenyl.

As used herein the term “alkynyl” can be straight-chain or branched unsaturated alkyl residues that contain one or more, for example one, two or three, triple bonds which can be in any suitable position. Of course, an unsaturated alkyl residue has to contain at least two carbon atoms. Examples of unsaturated alkyl residues are alkynyl residues such as ethynyl, 1-propynyl or propargyl.

As used herein the term “cycloalkyl” can be monocyclic or polycyclic, for example monocyclic, bicyclic or tricyclic, i.e., they can for example be monocycloalkyl residues, bicycloalkyl residues and tricycloalkyl residues, provided they have a suitable number of carbon atoms and the parent hydrocarbon systems are stable. A bicyclic or tricyclic cycloalkyl residue has to contain at least 4 carbon atoms. In an embodiment, a bicyclic or tricyclic cycloalkyl residue contains at least 5 carbon atoms. In a further embodiment, a bicyclic or tricyclic cycloalkyl residue contains at least 6 carbon atoms and up to the number of carbon atoms specified in the respective definition. Cycloalkyl residues can be saturated or contain one or more double bonds within the ring system. In particular they can be saturated or contain one double bond within the ring system. In unsaturated cycloalkyl residues the double bonds can be present in any suitable positions. Monocycloalkyl residues are, for example, cyclopropyl, cyclobutyl, cyclopentyl, cyclopentenyl, cyclohexyl, cyclohexenyl, cycloheptyl, cycloheptenyl, cyclooctyl, cyclononyl, cyclodecyl, cycloundecyl, cyclododecyl or cyclotetradecyl, which can also be substituted, for example by C₁-C₄ alkyl. Examples of substituted cycloalkyl residues are 4-methylcyclohexyl and 2,3-dimethylcyclopentyl. Examples of parent structures of bicyclic ring systems are norbornane, bicyclo[2.2.1]heptane, bicyclo[2.2.2]octane and bicyclo[3.2.1]octane.

As used herein, the term “aryl” means an aromatic substituent which is a single ring or multiple rings fused together. When formed of multiple rings, at least one of the constituent rings is aromatic. In an embodiment, aryl substituents include phenyl and naphthyl groups.

A novel class of chemical agents (i.e. novel hybrid molecules) having mixed vitamin D receptor agonism and histone deacetylase inhibitory properties are described herein.

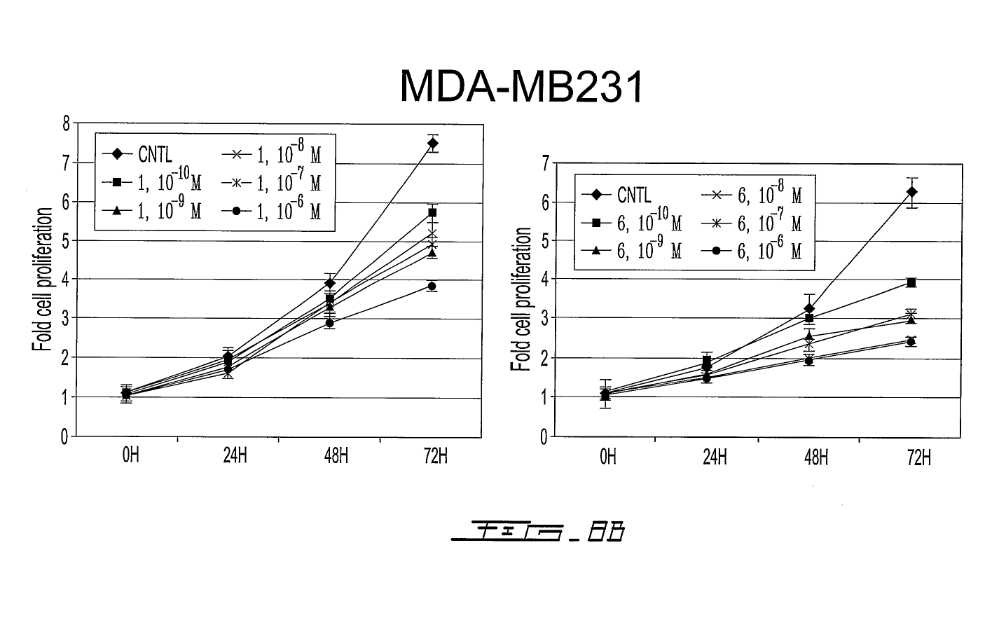

A combinatory effect of calcitriol and TSA in prostate and breast cancer has been previously demonstrated.¹⁰ Initial studies have shown a combinatory effect in low nM concentrations of TSA and calcitriol on the proliferation of calcitriol-resistant SCC4 HNSCC cells (FIG. 7). A hybrid molecule combining both vitamin D receptor agonism and HDAC inhibition properties into a single molecular structure was developed.

Hybrid molecules have had considerable success in pharmacotherapy and offer several advantages over the use of the individual compounds (i.e. the compounds making-up the hybrid molecule) in combination therapy.¹¹⁻¹³ Moreover, analyses of dose/toxicity relationships of hybrid molecules are simpler than those of combination therapies, and problems associated with differing pharmacokinetic profiles of individual components are eliminated.

The design of the hybrid molecules of the present invention is based on structure-activity relationship (SAR) and X-ray studies. In an embodiment of the present invention, the hybrid molecule is based on the structures of calcitriol and TSA. The crystal structure of calcitriol bound to the VDR-LBD reveals hydrogen bonding to all three hydroxyl functionalities (Ser237 and Arg274 for 1-OH; Ser278 and Tyr143 for 3-OH; and His305 and His397 for 25-OH).¹⁴ The remainder of the binding pocket is filled with hydrophobic residues which contact the triene and C/D-ring sections, as well as a portion of the side chain. The hydrogen bonding to the hydroxyl functionalities of the A-ring is critical for binding, as deletion or alteration of the stereochemistry of the 1- or 3-OH group significantly decreases affinity for the VDR.¹⁵ Most potent analogs of calcitriol have hydroxyl moieties in the vicinity of C-25, although some variation in their exact location (e.g. in EB1089, 4) is tolerated. The central C/D-ring is less critical, as it may be partially or fully excised in favor of a single 5- or 6-membered ring or a linear chain.^{16,17} 19-Nor and C-20 epi analogs are also well tolerated by the VDR.^{15,18}

The crystal structure of TSA (3) bound to an HDAC revealed a tube-like binding pocket possessing a zinc ion coordinated to two Asp residues and one His residue at a bottom portion of the tube-like binding pocket.¹⁹ The hydroxamic acid function of TSA forms a bidentate chelate with the zinc ion. There are two other Asp residues and two other His residues at the bottom portion of the tube-like binding pocket, the latter of which form hydrogen bonds with the NH and OH groups of the hydroxamic acid. The polyene chain of TSA spans the remainder of the tube-like binding pocket, consisting of hydrophobic residues. The top portion of the tube-like binding pocket terminates at a surface groove comprising several hydrophobic residues which come into contact with the dimethylamino group of TSA.

Based on SAR studies, the γ-methyl dienylhydroxamic acid unit is required. However, the ketone and adjacent methyl substituted methyne may be excised, provided that the dimethylamino group is replaced with a larger unit such as an arylsulfonamide.²⁰ Thus, the dienyl chain in TSA seems to function as a tether, linking the zinc binding unit with a “cap” group which binds on the HDAC surface. Hydrogenation of the dienyl chain in TSA analogs renders them inactive. However, straight chain analogs lacking the γ-methyl group (e.g. SAHA, 5) have been found to be potent HDAC inhibitors.

A first hybrid molecule (6) comprising the 3 hydroxyl moieties required for binding to the VDR was designed (Scheme 1). The backbone of the vitamin D core, including the A and C/D-ring systems were maintained along with the stereochemical relationships of the various substituents.

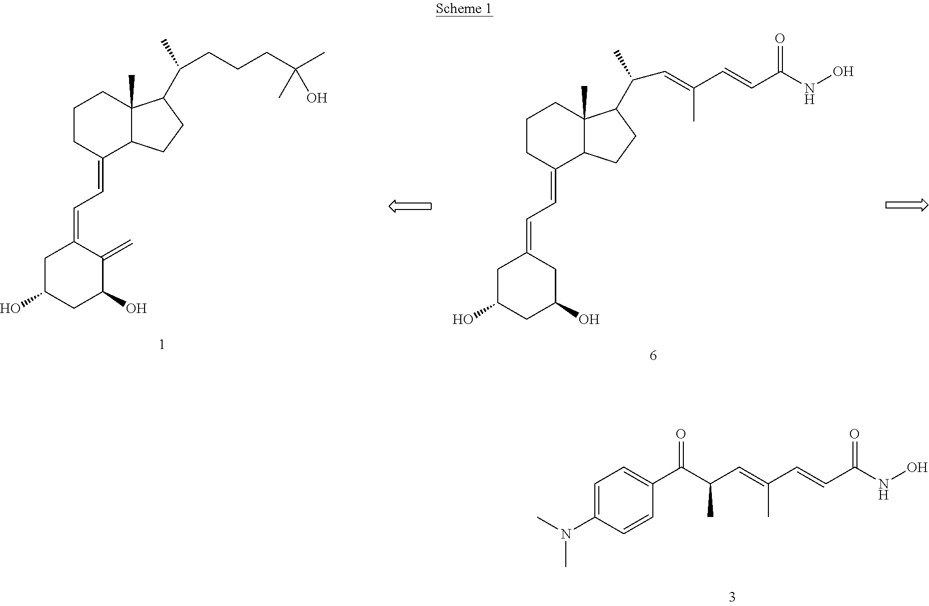

The typical side chain extending off the D-ring of vitamin D was replaced by a γ-methyl dienylhydroxamic acid unit characteristic of TSA. It was hypothesized that the hydroxamic acid terminus of this polar side chain would allow hydrogen bond formation in the active site of the VDR, as well as permitting chelation to the zinc ion in the HDAC binding site.

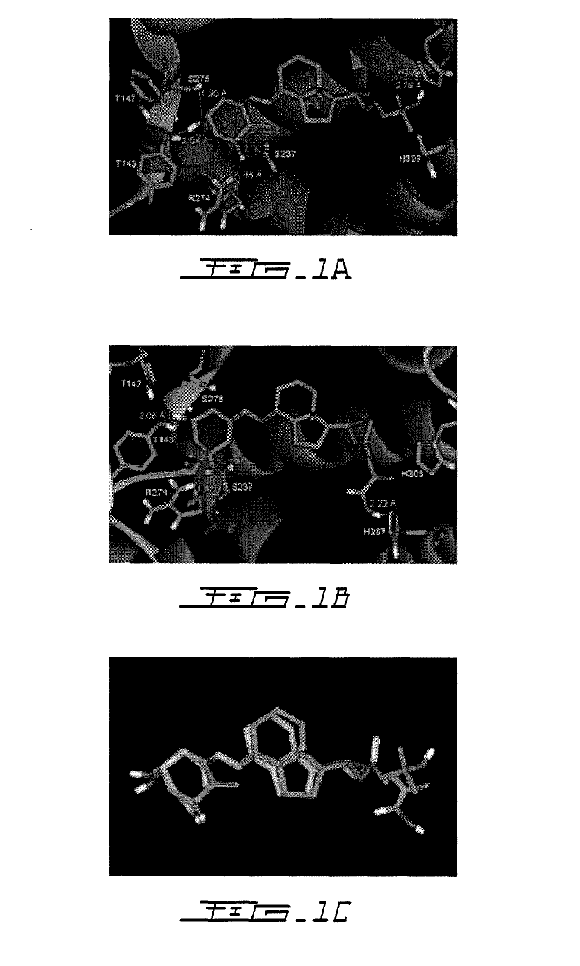

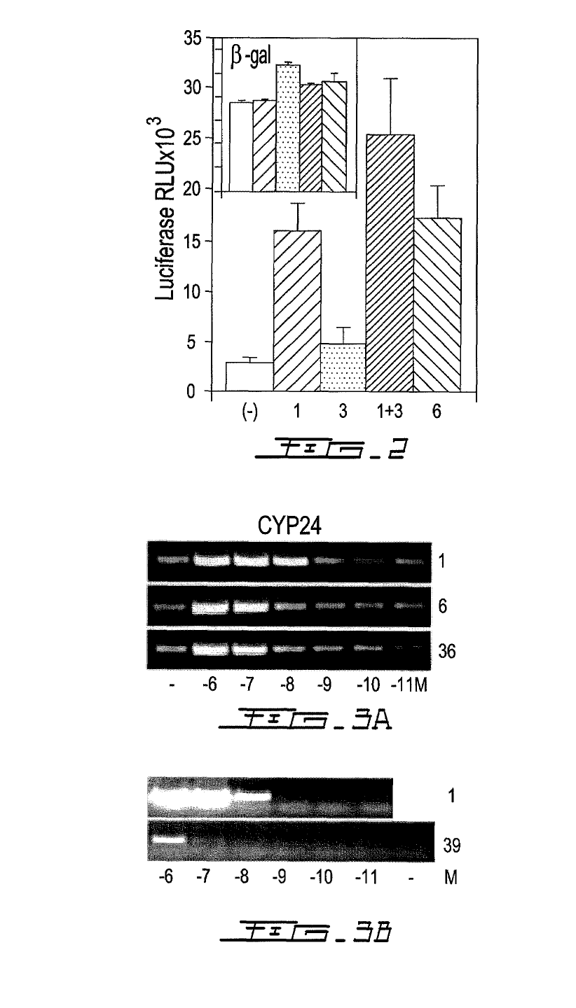

Preliminary docking experiments using AutoDoc 3.0® indicated that 6 should bind to the VDR in an orientation substantially similar to calcitriol. As can be observed from FIG. 1, the minimized structure retained the critical hydrogen bonds between the A-ring and the receptor (Ser237 and Arg274 for 1-OH; Ser278 and Tyr143 for 3-OH). Moreover, the hydroxamic acid is slightly twisted away from the location of the C-25 hydroxyl of calcitriol, preventing a bifurcated hydrogen bond from forming. However, there is a strong hydrogen bond between the hydroxamate OH and His397. The affinity of 6 for the receptor was computed to be between that of calcitriol and EB1089, both of which are effective ligands in several cancer cell lines.

In an embodiment, the present invention relates to pharmaceutical compositions comprising a pharmaceutically effective amount of one or more hybrid molecules as defined herein, or pharmaceutically acceptable salts thereof, in association with one or more pharmaceutically acceptable carriers, excipients and/or diluents. The term “pharmaceutically effective amount” is understood as being an amount of hybrid molecule required upon administration to a mammal in order to induce vitamin D receptor agonism and HDAC inhibition. Therapeutic methods comprise the step of treating patients in a pharmaceutically acceptable manner with one or more hybrid molecules or compositions comprising one or more hybrid molecules as disclosed herein. Such compositions may be in the form of tablets, capsules, caplets, powders, granules, lozenges, suppositories, reconstitutable powders, creams, lotions, or liquid preparations, such as oral or sterile parenteral solutions or suspensions.

The therapeutic agents of the present invention (i.e. hybrid molecules) may be administered alone or in combination with pharmaceutically acceptable carriers. The proportion of each carrier is determined by the solubility and chemical nature of the agent(s), the route of administration, and standard pharmaceutical practice. In order to ensure consistency of administration, in an embodiment of the present invention, the pharmaceutical composition is in the form of a unit dose. The unit dose presentation forms for oral administration may be tablets and capsules and may contain conventional excipients. Non-limiting examples of conventional excipients include binding agents such as acacia, gelatin, sorbitol, or polyvinylpyrolidone; fillers such as lactose, sugar, maize-starch, calcium phosphate, sorbitol or glycine; tabletting lubricants such as magnesium stearate; disintegrants such as starch, polyvinylpyrrolidone, sodium starch glycolate or microcrystalline cellulose; or pharmaceutically acceptable wetting agents such as sodium lauryl sulphate.

The hybrid molecules of the present invention may be injected parenterally; this being intramuscularly, intravenously, or subcutaneously. For parenteral administration, the hybrid molecules may be used in the form of sterile solutions containing solutes, for example sufficient saline or glucose to make the solution isotonic.

The hybrid molecules maybe administered orally in the form of tablets, capsules, or granules, containing suitable excipients such as starch, lactose, white sugar and the like. The hybrid molecules may be administered orally in the form of solutions which may contain coloring and/or flavoring agents. The hybrid molecules may also be administered sublingually in the form of tracheas or lozenges in which the active ingredient(s) is/are mixed with sugar or corn syrups, flavoring agents and dyes, and then dehydrated sufficiently to make the mixture suitable for pressing into solid form.

The solid oral compositions may be prepared by conventional methods of blending, filling, tabletting, or the like. Repeated blending operations may be used to distribute the active agent(s) (i.e. hybrid molecules) throughout those compositions employing large quantities of fillers. Such operations are, of course, conventional in the art. The tablets may be coated according to methods well known in normal pharmaceutical practice, in particular with an enteric coating.

Oral liquid preparations may be in the form of emulsions, syrups, or elixirs, or may be presented as a dry product for reconstitution with water or other suitable vehicle before use. Such liquid preparations may or may not contain conventional additives. Non limiting examples of conventional additives include suspending agents such as sorbitol, syrup, methyl cellulose, gelatin, hydroxyethylcellulose, carboxymethylcellulose, aluminum stearate gel, or hydrogenated edible fats; emulsifying agents such as sorbitan monooleate or acaci; non-aqueous vehicles (which may include edible oils), such as almond oil, fractionated coconut oil, oily esters selected from the group consisting of glycerine, propylene glycol, ethylene glycol, and ethyl alcohol; preservatives such as for instance methyl para-hydroxybenzoate, ethyl para-hydroxybenzoate, n-propyl parahydroxybenzoate, or n-butyl parahydroxybenzoate or sorbic acid; and, if desired, conventional flavoring or coloring agents.

For parenteral administration, fluid unit dosage forms may be prepared by utilizing one or more hybrid molecules and a sterile vehicle, and, depending on the concentration employed, the hybrid molecule(s) may be either suspended or dissolved in the vehicle. Once in solution, the hybrid molecule(s) may be injected and filter sterilized before filling a suitable vial or ampoule followed by subsequently sealing the carrier or storage package. Adjuvants, such as a local anesthetic, a preservative or a buffering agent, may be dissolved in the vehicle prior to use. Stability of the pharmaceutical composition may be enhanced by freezing the composition after filling the vial and removing the water under vacuum, (e.g., freeze drying). Parenteral suspensions may be prepared in substantially the same manner, except that the hybrid molecule(s) should be suspended in the vehicle rather than being dissolved, and, further, sterilization is not achievable by filtration. The hybrid molecule(s) may be sterilized, however, by exposing it to ethylene oxide before suspending it in the sterile vehicle. A surfactant or wetting solution may be advantageously included in the composition to facilitate uniform distribution of the hybrid molecule(s).

Topical administration can be used as the route of administration when local delivery of one or more hybrid molecules is desired at, or immediately adjacent to, the point of application of the composition or formulation comprising one or more hybrid molecules.

The pharmaceutical compositions of the present invention comprise a pharmaceutically effective amount of one or more hybrid molecules as described herein and one or more pharmaceutically acceptable carriers, excipients and/or diluents. In an embodiment of the present invention, the pharmaceutical compositions contain from about 0.1% to about 99% by weight of a hybrid molecule as disclosed herein. In a further embodiment of the present invention, the pharmaceutical compositions contain from about 10% to about 60% by weight of a hybrid molecule as disclosed herein, depending on which method of administration is employed. Physicians will determine the most-suitable dosage of the present therapeutic agents (i.e. hybrid molecules). Dosages may vary with the mode of administration and the particular hybrid molecule chosen. In addition, the dosage may vary with the particular patient under treatment. The dosage of the hybrid molecule used in the treatment may vary, depending on the condition, the weight of the patient, the relative efficacy of the compound and the judgment of the treating physician.

Synthesis of Hybrid Molecule 6

The C/D-ring of the vitamin D core of 6, complete with its stereochemical information, was conveniently prepared by oxidative degradation of vitamin D₂ (17). Attachment of the A-ring via a Horner coupling of the A-ring phosphine oxide (16) yielded the desired core backbone. Elongation of the side chain by a series of olefination reactions yielded a carbonyldienyl moiety which was subsequently converted into a dienylhydroxamic acid moiety.

The A-ring of phosphine oxide (16) was prepared from (−)-Quinic acid (7) as illustrated herein below in Scheme 2.

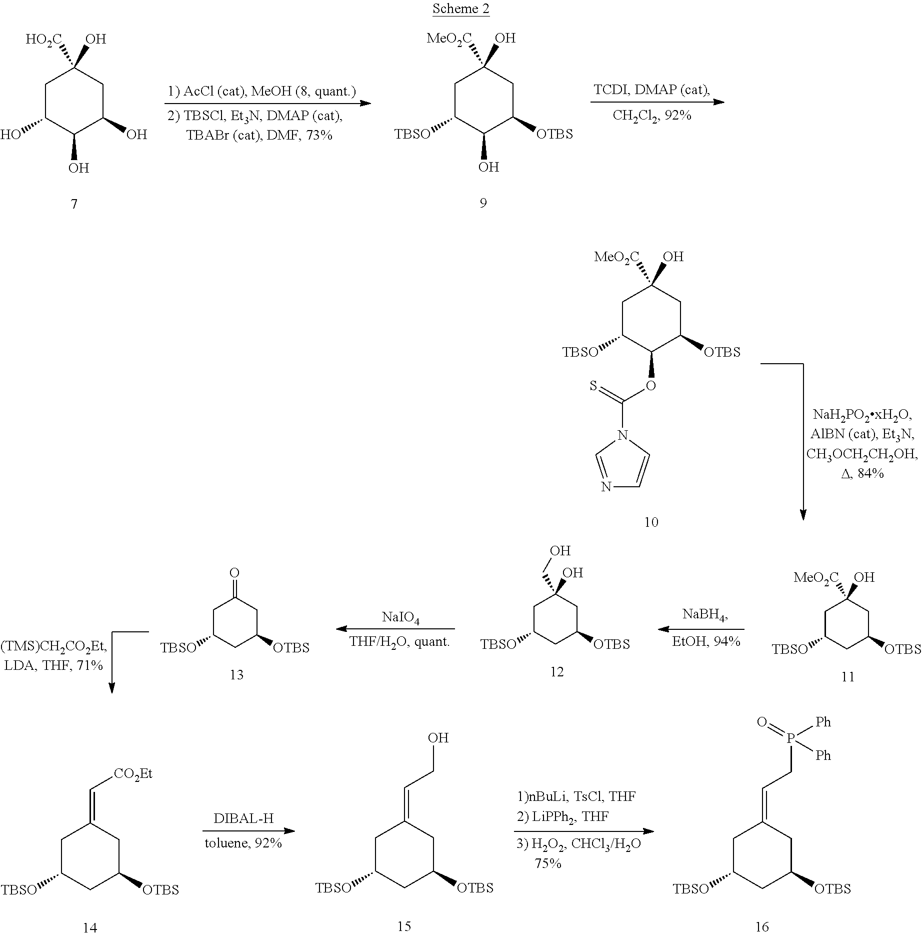

Thus, 7 was converted to the methyl ester 8 and then selectively bis-silylated on the less sterically hindered hydroxyls to provide 9 in 73% yield. Selective reaction of the secondary hydroxyl of 9 with 1,1-thiocarbonyldiimidazole (TCDI) provided intermediate 10 in excellent yield (92%). Radical deoxygenation of 10, using NaH₂PO₂ as the hydrogen atom source,²¹ provided 11 in good yields (84%). The reduction of ester 11 using NaBH₄, followed by oxidative cleavage of vicinal diol 12 using NaIO₄, provided ketone 13 in essentially quantitative yield.

Homologation of ketone 13 is difficult due to the ease of elimination of both β-siloxy substituents. The use of less nucleophilic reagents, particularly those as used in the phosphorous based olefination reactions, led to the undesired eliminations and subsequent aromatization to produce phenol. However, the use of the (TMS)CH₂CO₂Et/LDA reagent system under Peterson olefination conditions, provided the α,β-unsaturated ester 14 in good yield (71%). Ester 14 was subsequently reduced to the allylic alcohol 15 (92%) using DIBAL-H.

The allylic phosphine 16 was prepared from allylic alcohol 15 via the in situ formation of a tosylate, followed by displacement with LiPPh₂.²² Subsequent oxidation with aqueous hydrogen peroxide afforded the desired phosphine oxide 16 in 75% yield following recrystallization from methanol.

The core C/D-ring system of 6 was conveniently prepared by oxidative degradation (i.e. ozonolytic cleavage) of vitamin D₂ (17) in methanol using CHCl₃ as co-solvent (Scheme 3).

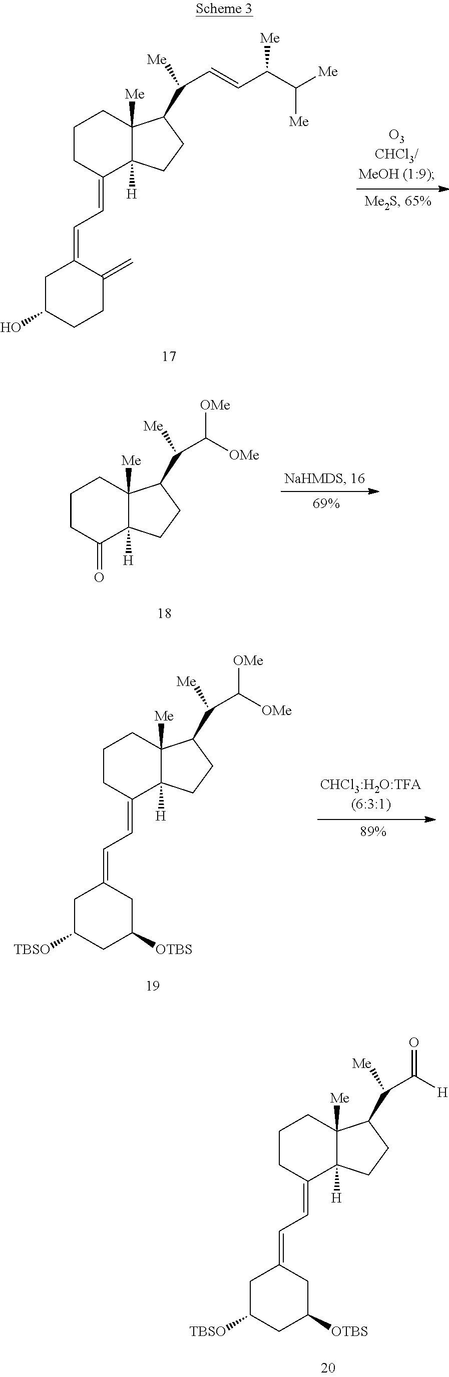

The residual acid in CHCl₃ was sufficient to catalyze acetalization of the aldehyde functionality of the in situ generated keto-aldehyde to provide, after a reductive quench with dimethylsulfide, intermediate 18 in 84% yield. In the absence of CHCl₃, the keto-aldehyde is isolated following a reductive quench of the ozonolysis. It is important to note that the acetal formation/reductive quench step must be carefully monitored by TLC, as epimerization of the C-14 stereocenter readily occurs. Indeed, if a stronger acid is used to catalyze acetal formation, epi-18 is obtained as the major product of the reaction. Horner coupling of phosphine oxide 16 with keto-acetal 18 provided the vitamin D backbone 19 in 69% yield. Acetal deprotection of 19 was achieved using a 6:3:1 mixture of CHCl₃:H₂O:TFA at 0° C. Although the deprotection step is slow at this temperature (3-4 h), careful monitoring of the reaction is required to avoid epimerization of the C-20 stereocenter.

E/Z selectivity in the subsequent olefination reactions extending the side arm is crucial as separation of the isomers would have been difficult, if at all possible. Wittig olefination of aldehyde 20 provided the α,β-unsaturated ester 21 in excellent yield (98%) and with >95:5 E:Z selectivity (Scheme 4).

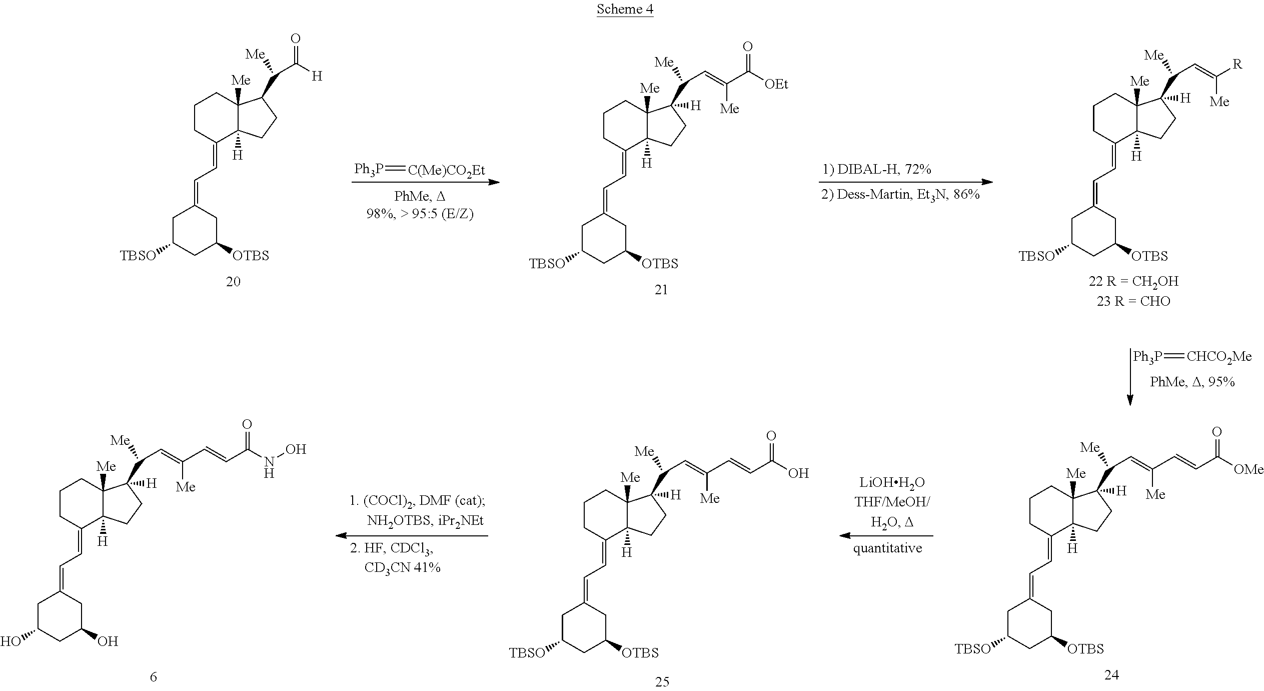

DIBAL-H reduction of the ester provided allylic alcohol 22 (72%), which was subsequently oxidized to aldehyde 23 (86%) using Dess-Martin periodinane in the presence of Et₃N. Oxidation in the absence of a weak base resulted in some deprotection of the A-ring hydroxyls due to the presence of residual acid in the Dess-Martin reagent. A second Wittig olefination provided the dienyl ester 24 (95%) with the newly generated double bond being exclusively of the E-configuration. Dienyl ester 24 was hydrolyzed to carboxylic acid 25 using LiOH in near quantitative yield. Acid 25 was transformed in situ to the acid chloride prior to treatment with O-(tert-butyldimethylsilyl)hydroxylamine to produce the tri-TBS-protected hydroxamic acid which was immediately deprotected using HF in acetonitrile. Hybrid molecule 6 was isolated in 41% yield from ester 24, as a white solid after purification by reverse-phase silica gel chromatography.

Synthesis of Hybrid Molecule 36

The preparation of 36, complete with its stereochemical information, is illustrated herein below in Scheme 5 starting by oxidative degradation of vitamin D₂ (17).

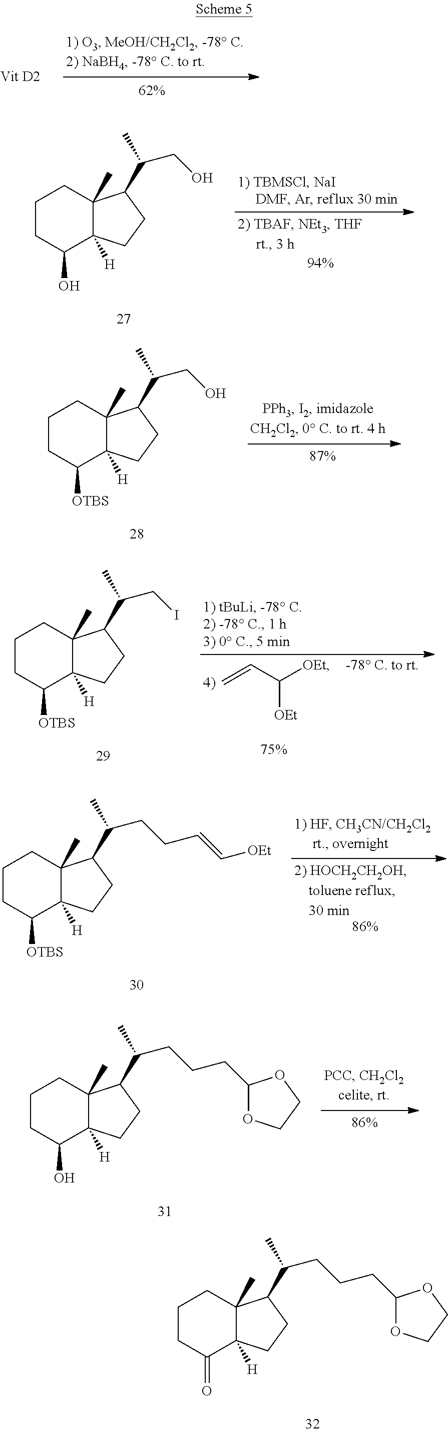

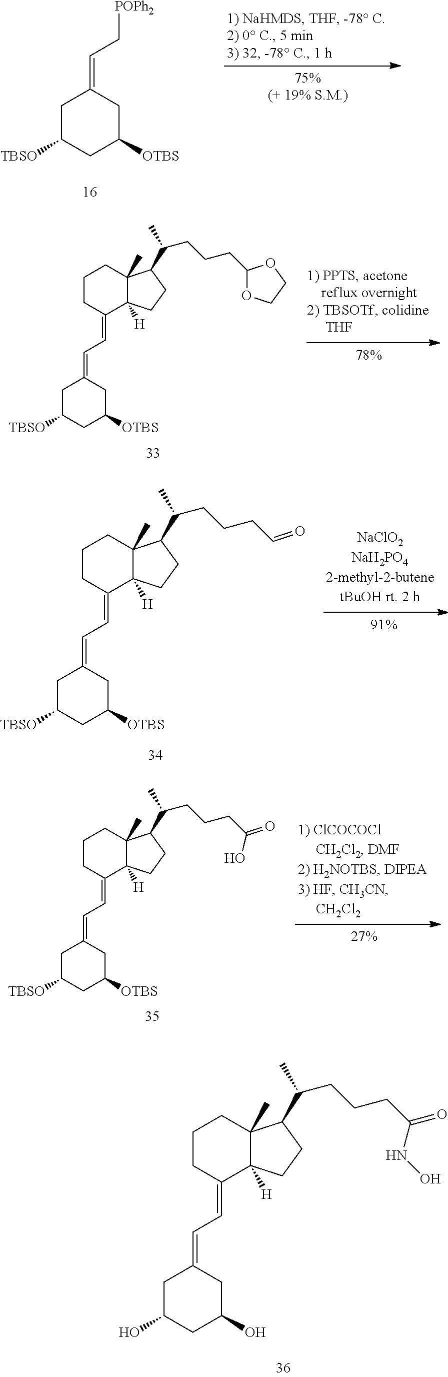

Synthesis of Hybride Molecule 39

The preparation of 39, complete with its stereochemical information is based on Scheme 5 as well as illustrated herein below in Scheme 6.

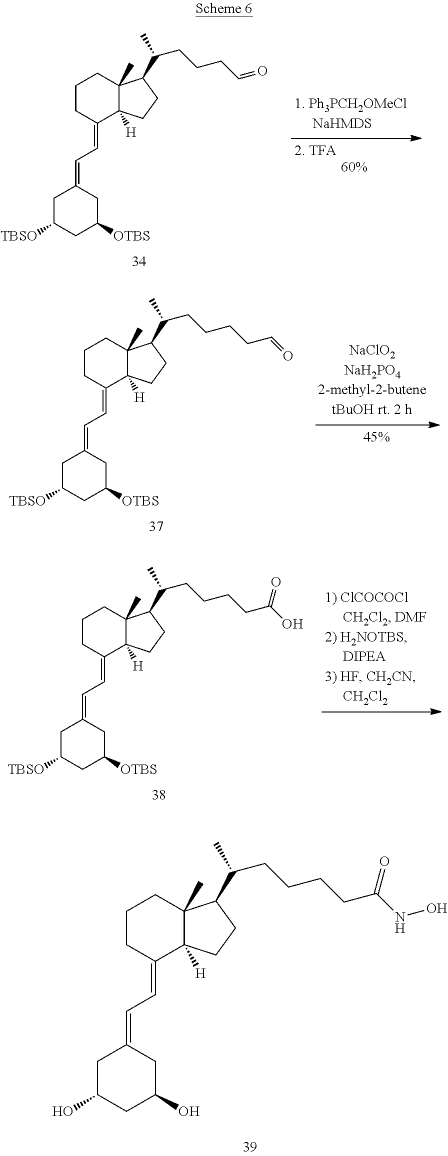

Biological Activity

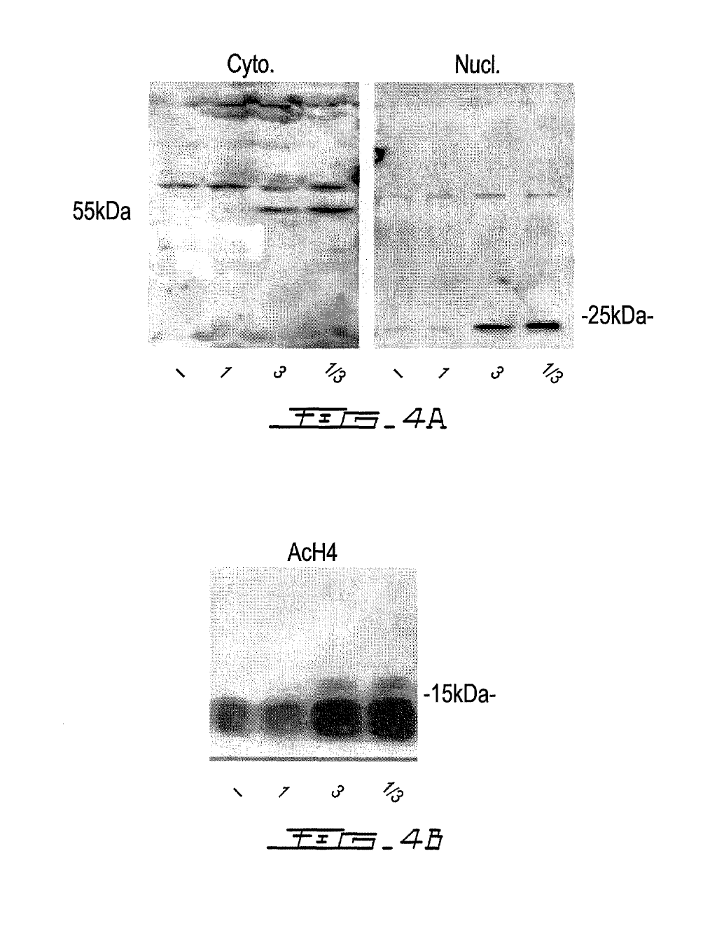

Hybrid molecule 6 was tested for calcitriol agonist activity using a reporter gene assay under standard conditions.^{25,26} COS7 cells were transiently co-transfected with a plasmid expression vector for the human VDR, a plasmid vector expressing bacterial β-galactosidase from a constitutively active promoter (as an internal control for transfection efficiency), and a vector containing a luciferase reporter gene, under control of a previously described synthetic promoter composed of three high affinity VDREs (Vitamin D Response Elements) placed immediately upstream of a truncated promoter region from the herpes simplex virus thymidine kinase gene.²⁵ Cells were left overnight in the presence of DNA and transfection reagent (lipofectamine). The media were changed and cells were incubated in fresh media containing either vehicle (DMSO), 1 (100 nM), 3 (15 nM), 1 and 3 combined (100 nM/15 nM) or 6 (100 nM), and incubated a further 36 h. As illustrated in FIG. 2, 3 alone had no substantial effect on reporter gene activity. Moreover, the calcitriol agonist activity of 6 is essentially identical to that of 1. The results were not normalized for β-galactosidase because TSA modestly affects expression from the internal control plasmid.

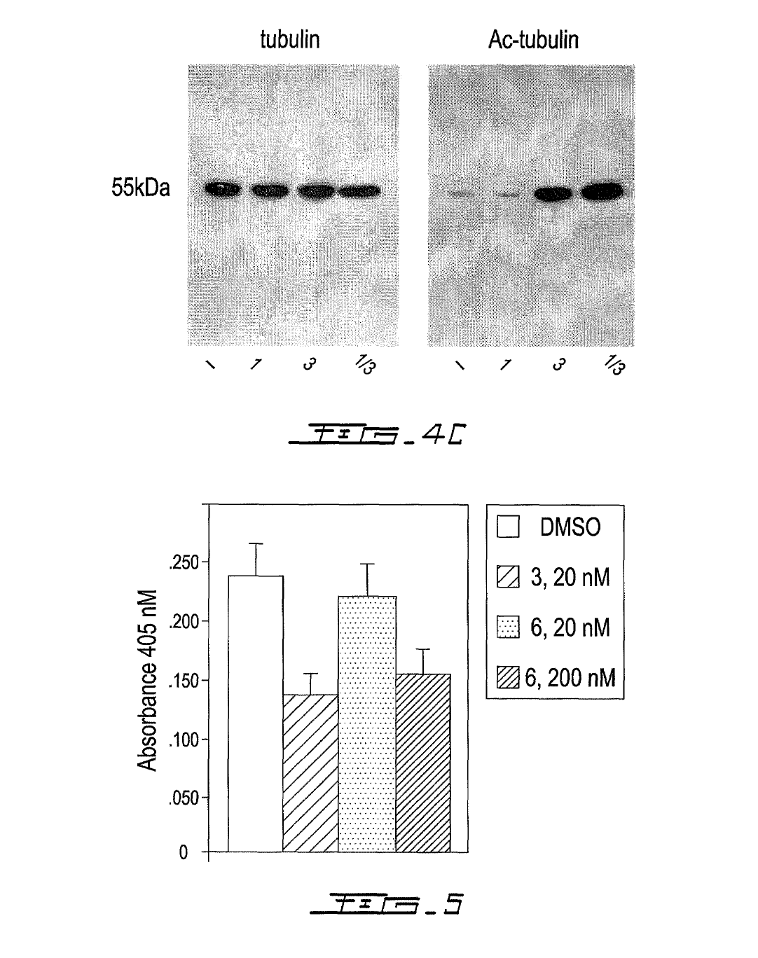

As illustrated in FIG. 3, hybrid molecules 6 and 36 along with 1 (A), and 39 along with 1 (B), were tested for VDR agonist activity in SCC4 cells treated for 24 h with either vehicle (−) or a range of concentrations of 1, 6, 36 or 36 from 10⁻¹¹ to 10⁻⁶M. The VDR agonist activity was assessed by analyzing induction of expression of the gene encoding CYP24 by reverse transcription/PCR.

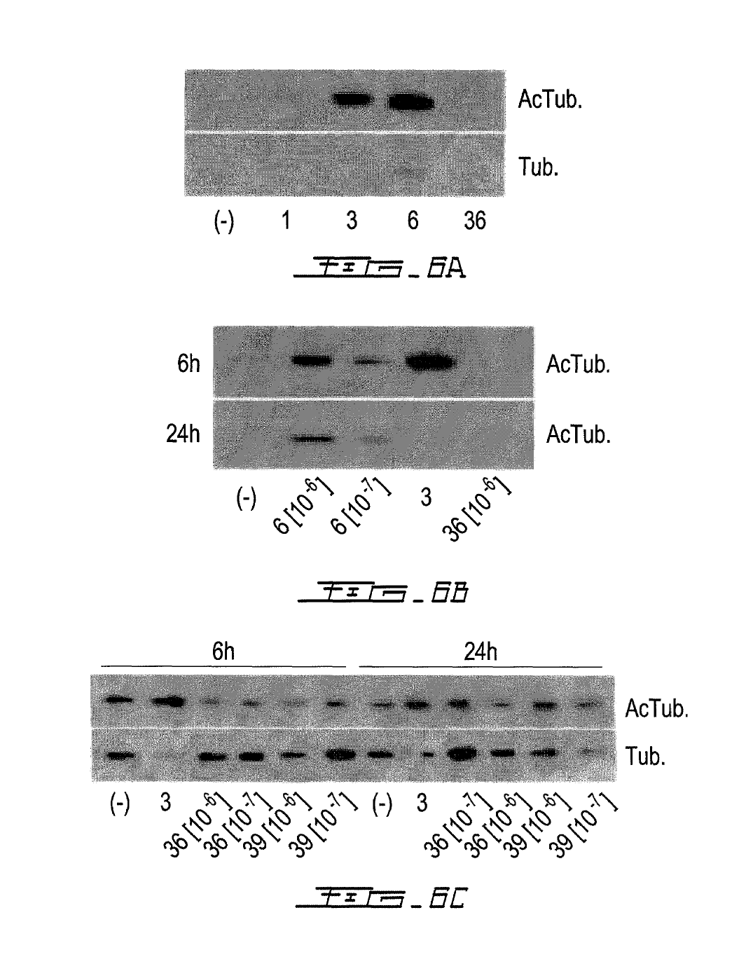

In preliminary experiments, TSA-inducible protein acetylation was analyzed in nuclear and cytoplasmic extracts of SCC4 cells treated for 6 h with 1, 3, or 1 and 3 and subsequently probed by Western blotting for protein acetylation using an anti-acetyllysine antibody. As expected, 3 markedly enhanced the acetylation of low molecular weight nuclear proteins that most likely corresponded to histones, whereas 1 had no effect alone or in combination with 3 (FIG. 4A, right panel). Effects of 3 on histone acetylation specifically, were confirmed by probing for acetylation of histone H4 (FIG. 4B). Similar to the results of FIG. 4A, 3 markedly enhanced histone H4 acetylation, while 1 had little effect on its own or in the presence of 3, indicating that 1 has little effect on global histone hyperacetylation. In the cytoplasmic extracts, it was observed that contrary to 3, 1 modestly but consistently enhanced the levels of a product of ˜70 kDa (FIG. 4A, left panel). However, it was not clear whether this corresponded to enhanced expression or acetylation. More strikingly, 3 markedly enhanced the acetylation of a 55 kDa protein, likely to be tubulin. This was confirmed by probing cytoplasmic fractions with an antibody that recognized α-tubulin (FIG. 4C). Calcitriol (1) had no effect on the acetylation of α-tubulin, and none of the treatments had any substantial effect on total α-tubulin expression (FIG. 4C).

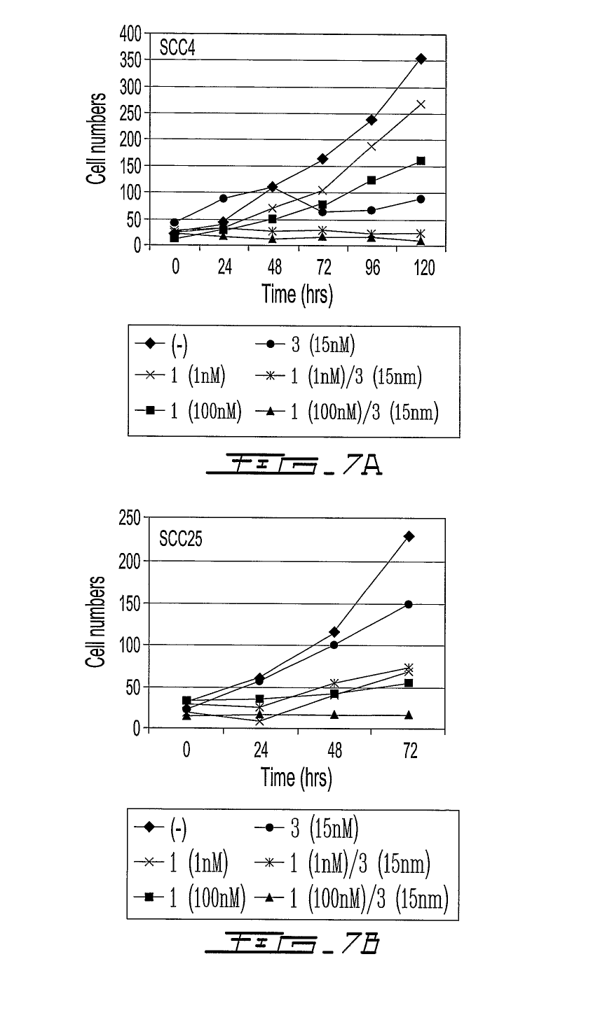

Hybrid molecule 6 was tested for HDAC inhibitory activity using a colorimetric assay as illustrated in FIG. 5. Nuclear extracts of SCC4 cells were incubated with vehicle, 3 or 6 in the presence of substrate for 60 min at 37° C. Following incubation with developer (1 min.), the absorbance was measured at 405 nM. The results showed that at equimolar concentrations (20 nM), 6 was less effective at inhibiting HDAC activity. However, when a ten-fold excess of 6 was employed, (200 nM) similar HDAC activity was observed indicating that 6 is about 10-fold less potent than TSA (3).

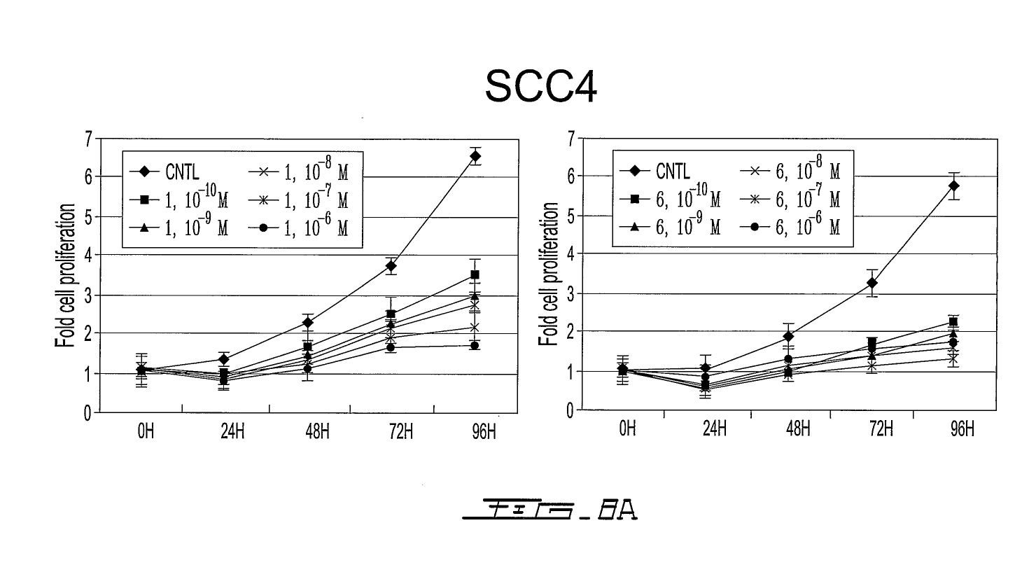

As illustrated in FIG. 6A, hybrid molecules 6 and 36 were also tested for HDAC inhibitory activity by determining their capacity to enhance acetylation of tubulin in SCC4 cells. SCC4 cells were incubated with vehicle or compound as indicated for 6 h and cytoplasmic protein extracts were probed by Western blotting using a specific antibody for acetylated α-tubulin. The results show that contrary to 36, 1 μM of 6 induces marked tubulin acetylation. As illustrated in FIG. 6B, SCC4 cells were treated for 6 or 24 h with 10⁻⁶ or 10⁻⁷M 6, 15 nM 3, or 10⁻⁶ M 36 and extracts were probed for levels of acetylated α-tubulin. Tubulin acetylation remains elevated in the presence of 6 after 24 h, whereas 36 had no effect on tubulin acetylation at either time point. As illustrated in FIG. 6C, SCC4 cells were incubated with vehicle or compound 3 (15 nM), 36 (10⁻⁶ or 10⁻⁷M) or 39 (10⁻⁶ or 10⁻⁷M) for 6 h or 24 h and protein extracts were probed for total alpha-tubulin (tub.) or acetylated alpha-tubulin (AcTub.) by Western blotting. Elevated levels of acetylated alpha-tubulin were observed only in cells treated with 3 when results were normalized for total alpha-tubulin.

In preliminary experiments, 3 alone completely inhibited SCC4 proliferation at concentrations of 50 nM, while higher concentrations resulted in substantial cell death (data not shown). As illustrated in FIG. 7A, cells were therefore treated with 15 nM 3 and either 1 nM 1 or 100 nM 1. As expected, either 1 nM 1 or 100 nM 1 had modest effects on SCC4 proliferation. However, the combination of low or high concentration of 1 with 15 nM 3 produced complete growth arrest. As illustrated in FIG. 7B, the more differentiated cell line SCC25 was relatively more sensitive to 1 at low or high concentrations and substantially less sensitive to 15 nM 3. Only the combination of 3 with 100 nM 1 produced completely blocked proliferation of SCC25 cells.

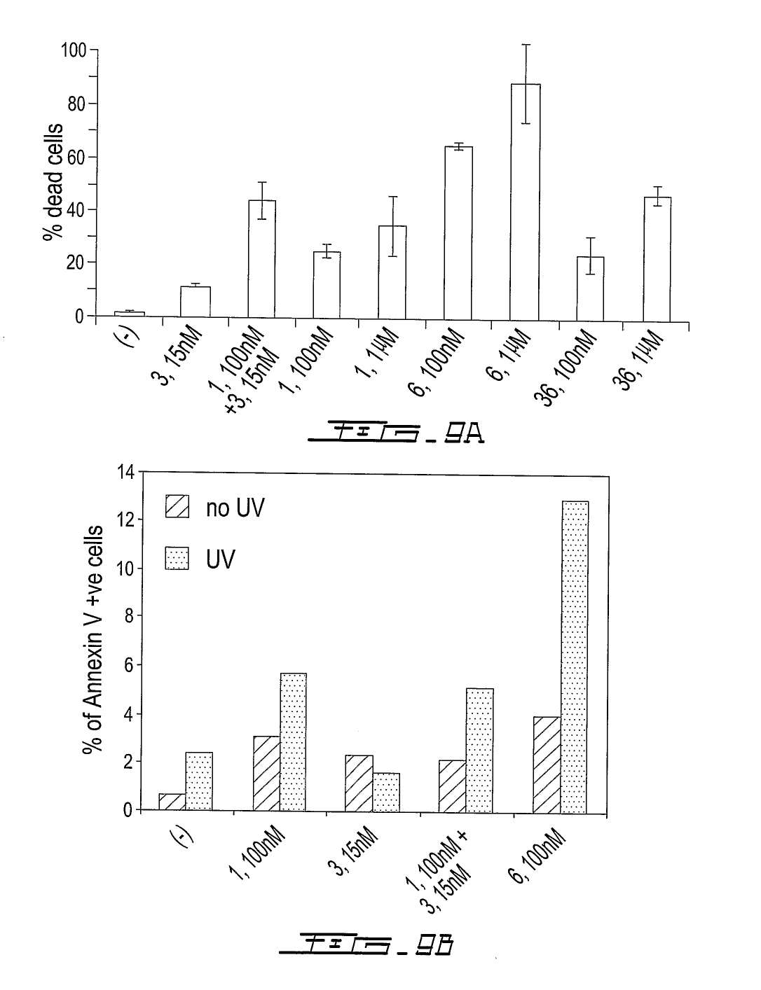

As illustrated in FIG. 8, hybrid molecule 6 was tested for its antiproliferative activity in the human cancer cell lines SCC4 and MDA-MB231. The SCC4 cell line (A) is representative of cells from advanced, de-differentiated squamous tumors and is resistant to the antiproliferative effects of 1 and its analogues.^{23,24} Subconfluent SCC4 cells were treated with vehicle (DMSO), 1 or 6 over a 96 h period. Tissue culture media was changed daily and fresh 1, 6 or vehicle were added. Under these conditions, 6 exhibited greater efficacy than 1 in inhibition of SCC4 proliferation. Similarly, treatment of the estrogen receptor negative breast cancer cell line MDA-MB231 (B), derived from a metastatic breast tumor, demonstrated similar potency and efficacy of 6. Data obtained in the prostate cancer cell lines PC3 and Du145 were similar but are not shown.

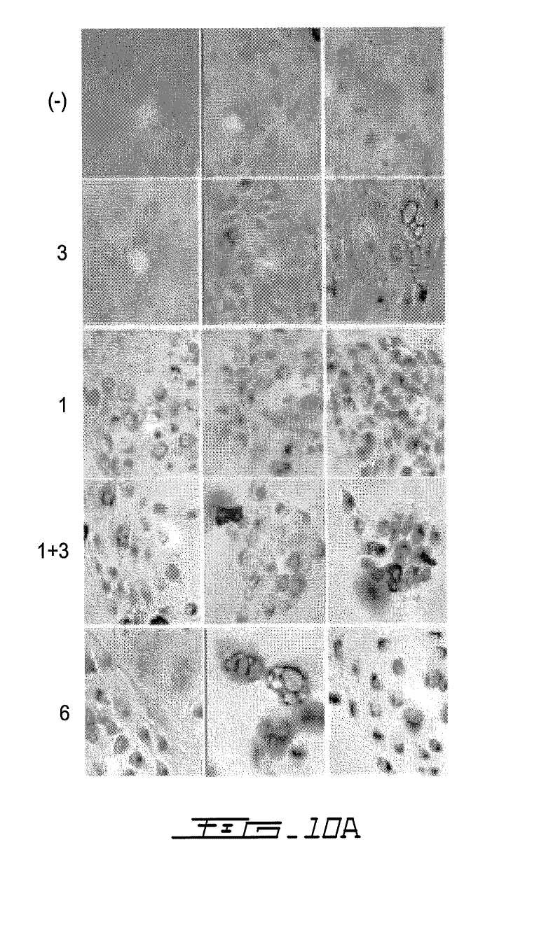

The effect of 1, 3, 1+3, 6 and 36 on cell viability was tested in two models. The viability of MCF-7 breast cancer cells was monitored after 24 h of incubation using a trypan blue dye exclusion assay (FIG. 9A). The results show that 6 induced markedly more cell death at concentrations of 10⁻⁷ or 10⁻⁶M than 1 at 10⁻⁶M or the combination of 1 and 3, whereas the effects of 36 were similar to those of 1. The effects of 100 nM 1, 15 nM 3 (TSA), 1 and 3 combined (100 nM/15 nM) and 100 nM 6 (hybrid molecule) on cell viability were tested by screening for induction of markers of apoptotic cell death. Annexin V staining was screened by FACS analysis as in Tavera-Mendoza et al.²⁶ While all treatments induced modest increases in annexin V staining, apoptosis could be excluded as the major cause of cell death (FIG. 9B). However, it was noted that 6 induced substantially higher levels of annexin V staining than 3, 1 or 1+3 after pretreatment with UV light, which sensitizes cells to apoptotic cell death.

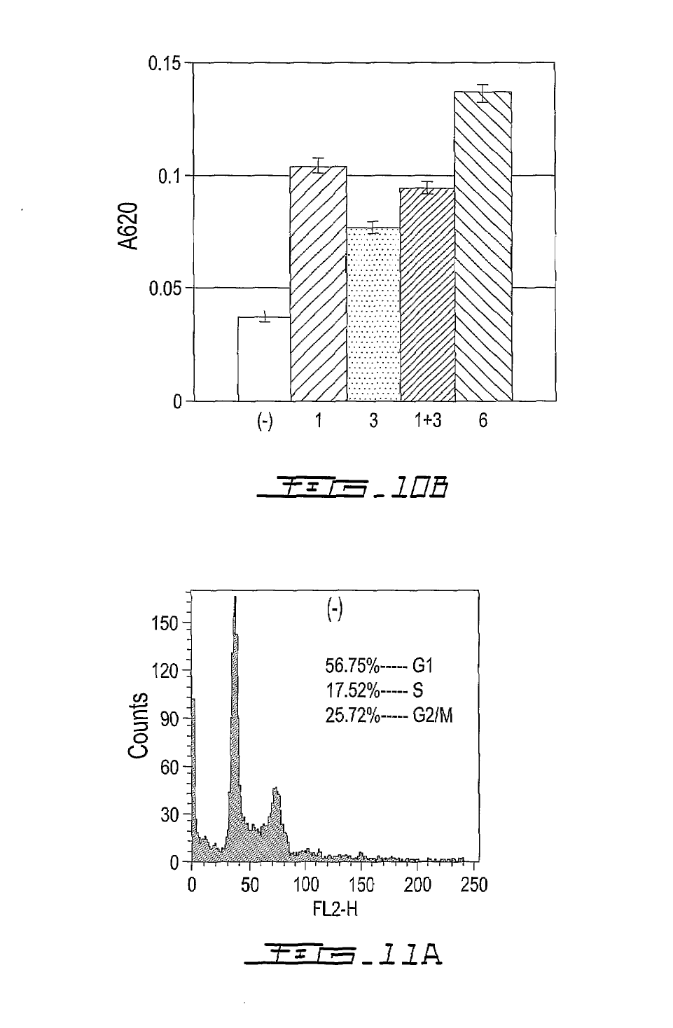

Since it was observed that treatment with 1 could enhance cell death by autophagy (ref. 26), the effects of different treatments on the induction of acidic β-galactosidase activity, a marker of autophagy (FIG. 10A) was tested. Cytochemical analysis of β-galactosidase expression by staining with 5-bromo-4-chloro-3-indoxyl-β-D-galactoside (X-Gal) at pH 4.0 in control (DMSO)-treated cells revealed none or very little staining. Treatment with 3 had weak or relatively modest effects on β-galactosidase levels. Treatment with 1 or 1+3 generally enhanced β-galactosidase staining, as did treatment with 6. While these are qualitative analyses, it was noted that treatment with 6 produced a higher percentage of cells that stained intensely for perinuclear β-galactosidase expression, characteristic of formation of autophagosomes. The effects of each treatment on the induction of acidic β-galactosidase expression was therefore assessed more quantitatively by spectrophotometric measurement of the indigo cleavage product of X-Gal at 620 nm.²⁷ These studies revealed that treatment with 6 induced ˜30% higher levels of β-galactosidase expression than treatment with 1 or 1+3 (FIG. 10B).

Taken together, the results illustrated in FIGS. 9 and 10 provide evidence that 6 displays enhanced biochemical activity over the combination of 1 and 3. As shown in FIG. 9, hybrid molecule 6 induced substantially higher levels of apoptosis as measured by annexin V staining than 1 and 3 in SCC4 squamous carcinoma cells sensitized for apoptotic cell death. As illustrated in FIG. 10, both cytochemical and quantitative analysis indicated that cells treated with 6 displayed elevated levels of autophagy in SCC4 cells, which also leads to cell death. These results suggest that hybrid molecules such as 6, which combine vitamin D agonism with HDAC inhibition, have enhanced therapeutic activity over combinations of vitamin D agonists and HDAC inhibitors.

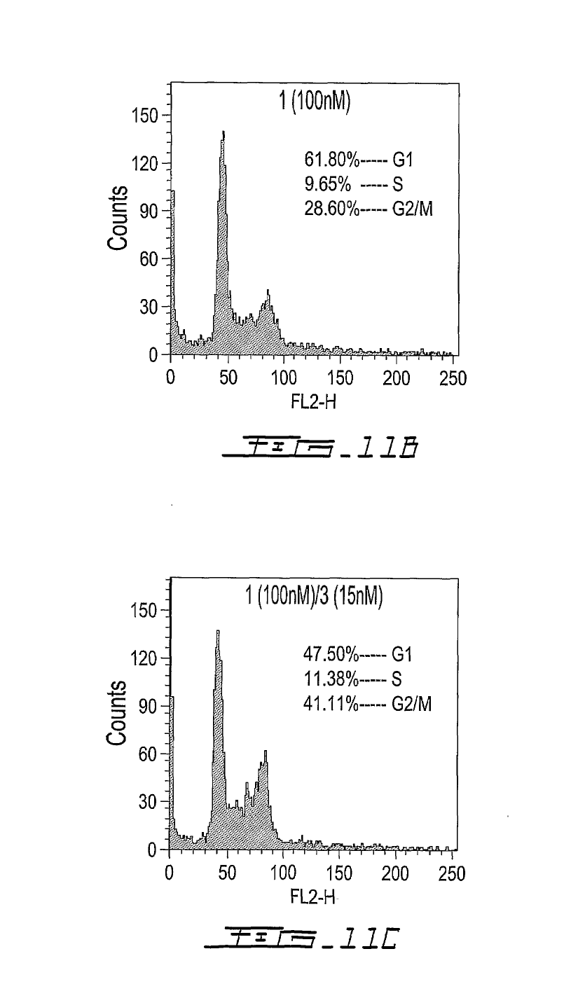

The combined effects of 1 and 3 on the distribution of SCC4 cells in the cell cycle were assessed by FACS analysis (FIG. 11). Cells were treated with vehicle, 100 nM 1, or a combination of 100 nM 1 and 15 nM 3 for 72 h, as indicated. Calcitriol (1) alone augmented the percentage of cells in either G1 or G2/M and substantially diminished the proportion of cells in S phase. While the combination of 1 and 3 diminished the proportion of cells in S phase, as expected, it also diminished the proportion of cells in G1 and markedly enhanced the number of cells in G2/M, indicative of G2/M arrest. This result differs from the G0/G1 arrest observed in SCC25 cells treated with 1 (ref. 27) but is consistent with other work showing that 3 alone inhibits cell proliferation at the G2/M checkpoint (ref. 28).

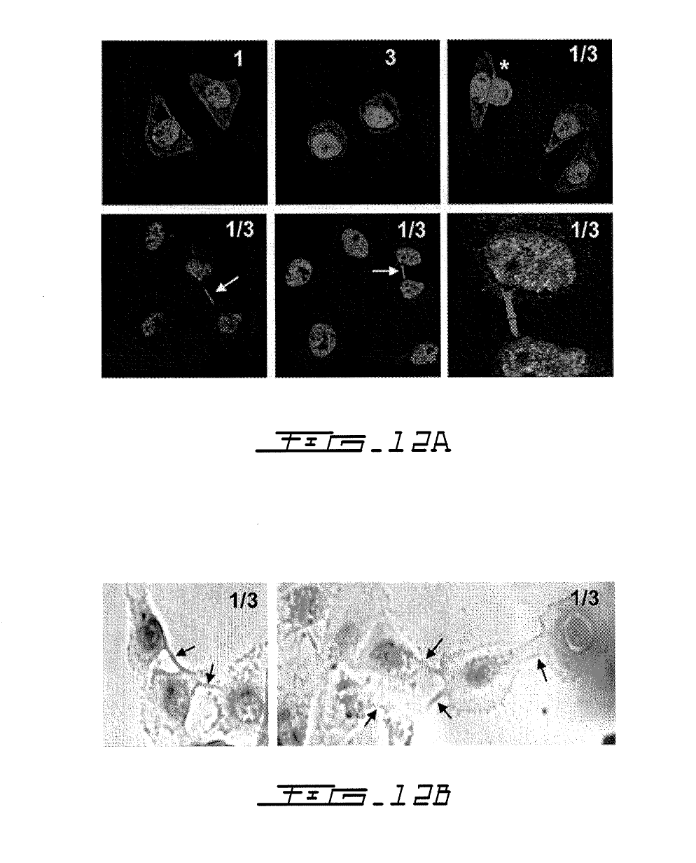

The combination of 1 and 3, but not 1 or 3 alone, induces mitotic catastrophe. Assembly into microtubules is regulated by acetylation. Tubulin deacetylation and destabilization is catalyzed by cytoplasmic HDAC6, whose activity can be blocked by 3 (ref. 29). Results obtained in cells treated with 1 and 3; such as the G2/M arrest, and the strong induction of tubulin acetylation and hence microtubule stabilization, suggest that the combination of 1 and 3 induces mitotic catastrophe in SCC4 cells. Previous studies have shown that microtubule stabilizing agents can induce mitotic catastrophe (ref. 30). SCC4 cells treated with either 1 or 3 individually were morphologically similar (FIG. 12A) and did not differ from control cells (not shown). In contrast, combined treatment produced morphological changes, including variations in cell size and shape, asymmetric cell divisions (FIG. 12A, asterisk), and the formation of intercellular microtubular bridges (FIG. 12A, arrows) reminiscent of telophase spindles (ref. 31). The formation of intercellular microtubular structures was consistent with observations in the light microscope of numerous intercellular bridges in cells treated with both 1 and 3 (FIG. 12B, arrows), but not in other treatment groups (not shown).

### Experimental

General. MeCN, toluene and CH₂Cl₂ were distilled from CaH₂ under argon. THF and Et₂O were distilled from sodium metal/benzophenone ketyl under argon. All other commercial solvents and reagents were used as received from the Aldrich Chemical Company, Fischer Scientific Ltd., EMD Chemicals Inc., Strem or BDH. All glassware was flame dried and allowed to cool under a stream of dry argon.

Silica gel (60 Å, 230-400 mesh) used in flash column chromatography was obtained from Silicycle and was used as received. Analytical thin-layer chromatography (TLC) was performed on pre-coated silica gel plates (Ultra Pure Silica Gel Plates purchased from Silicycle), visualized with a Spectroline UV₂₅₄ lamp, and stained with a 20% phosphomolybdic acid in ethanol solution, or a basic solution of KMnO₄. Solvent systems associated with R_{f} values and flash column chromatography are reported as percent by volume values.

¹H and ¹³C NMR, recorded at 300 MHz and 75 MHz respectively, were performed on a Varian Mercury 300 spectrometer. ¹H and ¹³C NMR, recorded at 400 MHz and 100 MHz respectively, were performed on a Varian Mercury 400 spectrometer. Proton chemical shifts were internally referenced to the residual proton resonance in CDCl₃ (δ 7.26 ppm), CD₃OD (δ 3.31 ppm), CD₃CN (δ 1.94 ppm), or d₆-DMSO (δ 2.50 ppm). Carbon chemical shifts were internally referenced to the deuterated solvent signals in CDCl₃ (δ 77.2 ppm), CD₃OD (δ 49.0 ppm), CD₃CN (δ 118.3 ppm and 1.3 ppm) or d₆-DMSO (δ 39.5 ppm). FT-IR spectra were recorded on a Nicolet Avatar 360 ESP spectrometer with samples loaded as neat films on NaCl plates. References following compound names indicate literature articles where ¹H and ¹³C NMR data have previously been reported.

### Methyl (3R,5R)-1,3,4,5-tetrahydroxycyclohexanecarboxylate (8).²¹

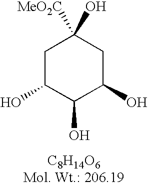

AcCl (2.044 g, 1.850 mL, 26.02 mmol) was added to a stirring solution of MeOH (37 mL) in a flame dried, round bottom flask charged with argon at 0° C. (−)-Quinic acid (7, 10.00 g, 52.04 mmol) was added to the mixture, and the suspension stirred for 16 h while warming to room temperature. The solid reactant dissolved as the reaction proceeded to afford a pale yellow solution. The reaction mixture was concentrated in vacuo, the residue redissolved in CHCl₃, and then concentrated again (this process was repeated three times to remove the excess MeOH via azeotropic distillation). The product was isolated as a viscous yellow oil in quantitative yield (10.80 g, 52.38 mmol). R_{f}=0.10 (30% EtOAc in hexanes); ¹H NMR (300 MHz, CD₃CN) δ 4.04-3.97 (1H, m), 3.96-3.84 (1H, m), 3.65 (3H, s), 3.34-3.26 (1H, m), 2.09-1.88 (3H, m), 1.74-1.63 (1H, m), 4 exchangeable protons unobserved; ¹³C NMR (75 MHz, CD₃CN) δ 175.0, 76.7 (2C), 71.3, 67.6, 53.0, 42.1, 38.0.

### Methyl (3R,5R)-3,5-bis[tert-butyl(dimethyl)silyloxy]-1,4-di-hydroxycyclohexanecarboxylate (9).²¹

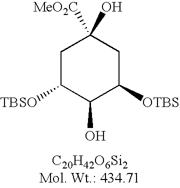

Compound 8 (10.73 g, 52.04 mmol) was dissolved in DMF (200 mL), in a flame dried round bottom flask flushed with argon. To this stirring solution was added DMAP (0.6358 g, 5.204 mmol), TBABr (1.730 g, 5.204 mmol), and TBSCI (17.26 g, 114.5 mmol). The flask was sealed with a rubber septum and cooled to 0° C., at which point Et₃N (11.85 g, 16.30 mL, 117.1 mmol) was added to the reaction via syringe. A fine white precipitate formed upon addition of the amine. The reaction was stirred under argon for 16 h while warming to room temperature. The reaction mixture was then filtered to remove the precipitate, the filtrate diluted with EtOAc (200 mL) and extracted with sat. NH₄Cl (3×100 mL), distilled H₂O (100 mL) and brine (100 mL). The organic layer was then separated, dried (MgSO₄), and concentrated in vacuo to provide the crude product as a yellow, viscous oil. Compound 9 was isolated as a fluffy white solid via FCC (30% EtOAc in hexanes) in 73% yield (16.42 g, 37.76 mmol). R_{f}=0.60 (30% EtOAc in hexanes); ¹H NMR (400 MHz, CDCl₃) δ 4.52 (1H, br s), 4.36 (1H, dt, J=4.5, 2.5 Hz), 4.11 (1H, ddd, J=13.0, 8.5, 4.5 Hz), 3.76 (3H, s), 3.42 (1H, dt, J=8.5, 2.5 Hz), 2.32 (1H, d, J=2.5 Hz), 2.18 (1H, ddd, J=13.0, 4.5, 2.5 Hz), 2.09 (1H, dd, J=14.0, 2.5 Hz), 2.01 (1H, ddd, J=14.0, 4.5, 2.5 Hz), 1.82 (1H, dd, J=13.0, 10.5 Hz), 0.90 (18H, d, J=6.0 Hz), 0.15 (6H, d, J=7.0 Hz), 0.11 (6H, d, J=5.0 Hz); ¹³C NMR (100 MHz, CDCl₃) δ 173.8, 76.3, 76.1, 71.6, 68.7, 52.8, 42.8, 38.0, 26.1 (6C), 18.4 (2C), −4.0, −4.3, −4.4, −4.7.

### Methyl (3R,5R)-3,5-bis[tert-butyl(dimethyl)silyloxy]-1-hydroxyl-4-[(1H-imidazol-1-ylcarbonothioyl)oxy]cyclohexanecarboxylate (10).²¹

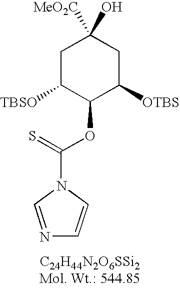

Compound 9 (5.534 g, 12.73 mmol) was dissolved in CH₂Cl₂ (14 mL) in a flamed dried round bottom flask. To this stirring solution was added DMAP (0.1555 g, 1.273 mmol) and TCDI (3.402 g, 19.09 mmol) which dissolved into solution after several hours of stirring. The reaction vessel was sealed with a rubber septum and flushed with argon, and the reaction mixture stirred at room temperature for 3 days. The reaction mixture was then concentrated to provide the crude product as a dark orange viscous oil, which was directly loaded on the silica gel. Compound 10 was isolated as a pale yellow viscous oil via FCC (gradient 30% to 50% EtOAc in hexanes) in 92% yield (6.401 g, 11.75 mmol). R_{f}=0.20 (30% EtOAc in hexanes); ¹H NMR (300 MHz, CDCl₃) δ 8.28 (1H, s), 7.54 (1H, s), 6.95 (1H, s), 5.43 (1H, dd, J=8.5, 3.0 Hz), 4.61-4.54 (2H, m), 4.50-4.41 (1H, m), 3.70 (3H, s), 2.27-2.13 (2H, m), 2.04-1.92 (2H, m), 0.82 (9H, s), 0.70 (9H, s), 0.01 (3H, s), 0.00 (3H, s), −0.05 (3H, s), −0.17 (3H, s); ¹³C NMR (75 MHz, CDCl₃) δ 183.6, 173.2, 137.0, 130.7, 117.7, 86.0, 75.1, 68.3, 65.4, 52.7, 43.0, 38.0, 25.8, 25.7 (2C), 25.5 (2C), 25.4, 17.8, 17.7, −4.2, −4.7, −4.9, −5.6.

### Methyl (3S,5S)-3,5-bis[tert-butyl(dimethyl)silyloxy]-1-hydroxy-cyclohexanecarboxylate (11).²¹

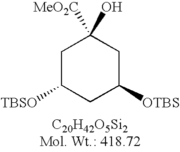

Compound 10 (9.490 g, 17.42 mmol) and NaH₂PO₂.×H₂O (7.660 g, 87.09 mmol) were dissolved in 2-methoxy-ethanol (230 mL) under argon in a flamed dried round bottom flask equipped with a reflux condenser, and heated to reflux using a heating mantle. In a separate flask, AIBN (0.5714 g, 3.484 mmol) was dissolved in 2-methoxy-ethanol (20 mL) and Et₃N (approx. 2 mL) was added to this solution until a pH of 8 was obtained. Half of the AIBN solution was added to the refluxing reaction mixture. The reaction was refluxed for 3 h with addition of the second half of the AIBN solution after 1 h. The reaction mixture was then cooled to room temperature, diluted with EtOAc (200 mL) and extracted with sat. NH₄Cl (3×100 mL), distilled H₂O (100 mL) and brine (100 mL). The organic layer was separated, dried (MgSO₄), and concentrated in vacuo to provide the crude product as a clear viscous oil. Compound 11 was isolated as a white solid via FCC (30% EtOAc in hexanes) in 84% yield (6.110 g, 14.59 mmol). R_{f}=0.60 (30% EtOAc in hexanes); ¹H NMR (300 MHz, CDCl₃) δ 4.76 (1H, s), 4.43-4.37 (1H, m), 4.32 (1H, tt, J=11.0, 4.5 Hz), 3.76 (3H, s), 2.25-2.16 (1H, m), 2.09-1.99 (1H, m), 1.97-1.92 (2H, m), 1.71 (1H, dd, J=13.0, 11.0 Hz), 1.51-1.42 (1H, m), 0.90 (9H, s), 0.88 (9H, s), 0.12 (3H, s), 0.10 (3H, s), 0.07 (6H, s); ¹³C NMR (75 MHz, CDCl₃) δ 174.3, 70.0, 63.7, 52.8, 44.9, 42.4, 39.7, 38.6, 26.2 (3C), 26.0 (3C), 18.5, 18.1, −4.2, −4.3, −4.7, −4.8.

### (3S,5S)-3,5-bis[tert-butyl(dimethyl)silyloxy]-1-(hydroxylmethyl)cyclohexanol (12).²¹

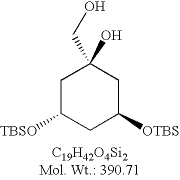

Compound 11 (6.110 g, 14.59 mmol) was dissolved in EtOH (150 mL) in a round bottom flask, and cooled to 0° C. NaBH₄ (1.656 g, 43.78 mmol) was added to the stirring solution. After 30 min of stirring at 0° C., the reaction mixture was warmed to room temperature and stirred overnight. The reaction mixture was then quenched with sat. NH₄Cl (50 mL) and diluted with EtOAc (100 mL). The layers were separated and the aqueous layer extracted with EtOAc (2×50 mL). The combined organic layers where further extracted with sat. NH₄Cl (2×50 mL), distilled H₂O (50 mL) and brine (50 mL), separated, dried (MgSO₄), and concentrated in vacuo to give the crude product as a translucent grey solid in 94% yield (5.368 g, 13.74 mmol). The diol 12 was carried forward without further purification. R_{f}=0.40 (30% EtOAc in hexanes); ¹H NMR (300 MHz, CDCl₃) δ 4.58 (1H, s), 4.42-4.26 (2H, m), 3.44-3.28 (2H, m), 2.21 (1H, dd, J=8.5, 4.5 Hz), 2.10-1.85 (3H, m), 1.50-1.36 (2H, m), 1.27 (1H, dd, J=12.5, 11.0 Hz), 0.92 (9H, s), 0.90 (9H, s), 0.13 (3H, s), 0.12 (3H, s), 0.09 (6H, s); ¹³C NMR (75 MHz, CDCl₃) δ 74.7, 71.0, 70.0, 64.2, 44.1, 43.0, 38.1, 26.2 (3C), 25.1 (3C), 18.4, 18.0, −4.3, 4.4, −4.7, −5.0.

### (3S,5S)-3,5-bis[tert-butyl(dimethyl)silyloxy]-cyclohexanone (13).²¹

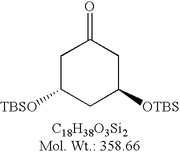

To a stirring solution of 12 (5.368 g, 13.74 mmol) in THF (100 mL), cooled to 0° C., was added an aqueous solution of NaIO₄ (4.408 g, 20.61 mmol) (50 mL). A fine, white precipitate formed as the reaction proceeded. The reaction mixture was then warmed to room temperature and stirred over night. The reaction mixture was then diluted with distilled H₂O until all of the precipitate dissolved. The layers were separated and the aqueous layer extracted with EtOAc (2×50 mL). The organic layers were combined and extracted with sat. NH₄Cl (2×50 mL), distilled H₂O (50 mL) and brine (50 mL), then dried (MgSO₄), and concentrated in vacuo to provide the crude product as a white crystalline solid in quantitative yield (4.938 g, 13.77 mmol). The ketone 13 was carried forward without further purification. R_{f}=0.40 (10% EtOAc in hexanes); ¹H NMR (300 MHz, CDCl₃) δ 4.34 (2H, m), 2.55 (2H, dd, J=14.0, 4.0 Hz), 2.35 (2H, ddd, J=14.0, 7.0, 1.0 Hz), 1.94 (2H, t, J=5.5 Hz), 0.87 (18H, s), 0.07 (6H, s), 0.06 (6H, s); ¹³C NMR (75 MHz, CDCl₃) δ 207.7, 67.0 (2C), 50.4 (2C), 42.3, 25.9 (6C), 18.2 (2C), −4.6 (2C), −4.7 (2C).

### Ethyl ((3R,5R)-3,5-bis[tert-butyl(dimethyl)silyloxy]-cyclohexylidene)acetate (14).²¹

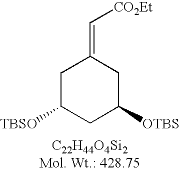

In a flame dried round bottom flask cooled to −78° C. under argon, n-BuLi (8.610 mmol) was added to a solution of i-Pr₂NH (0.8712 g, 8.610 mmol) in THF (100 mL). The mixture was suspended above the ice bath for 15 min, then recooled to −78° C. Ethyl-(trimethylsilyl)acetate (1.656 g, 10.33 mmol) was added to the stirring reaction mixture, and the reaction vessel was again suspended above the ice bath for 15 min and recooled to −78° C. Finally, a solution of 13 (2.471 g, 6.888 mmol) in THF (30 mL) was slowly cannulated into the reaction flask over a period of 30 min. The reaction mixture was then stirred at −78° C. for another 3 h, quenched with sat. NH₄Cl (50 mL) and warmed to room temperature. The layers were separated and the aqueous layer extracted with EtOAc (3×50 mL). The combined organic layers were extracted with distilled H₂O (50 mL) and brine (50 mL), dried (MgSO₄), and concentrated in vacuo to provide the crude product. Compound 14 was isolated as a clear oil via FCC (gradient 10% to 20% EtOAc in hexanes) in 71% yield (2.103 g, 4.905 mmol). R_{f}=0.70 (10% EtOAc in hexanes); ¹H NMR (300 MHz, CDCl₃) δ 5.70 (1H, s), 4.20-4.08 (4H, m), 3.06 (1H, dd, J=13.5, 6.0 Hz), 2.79 (1H, dd, J=13.5, 3.5 Hz), 2.40 (1H, dd, J=13.0, 3.5 Hz), 2.16 (1H, dd, J=13.0, 8.0 Hz), 1.88-1.78 (1H, m), 1.76-1.66 (1H, m), 1.28 (3H, t, J=7.0 Hz), 0.88 (9H, s), 0.86 (9H, s), 0.06 (12H, m); ¹³C NMR (75 MHz, CDCl₃) δ 166.4, 156.7, 117.5, 68.2, 68.1, 59.8, 46.3, 43.4, 37.7, 26.1 (3C), 26.0 (3C), 18.4, 18.3, 14.6, −4.5 (2C), −4.7 (2C).

### 2-((3R,5R)-3,5-bis[tert-butyl(dimethyl)silyloxy]cyclohexyl-idene) ethanol (15).²¹

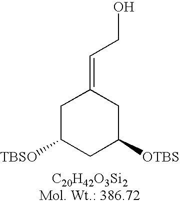

In a flame dried round bottom flask, cooled to −78° C. under argon, DIBAL-H (12.26 mmol) was added to a solution of 14 (2.103 g, 4.905 mmol) in toluene (50 mL). The reaction mixture was warmed to room temperature and stirred for another 3 h. The reaction mixture was then cooled to 0° C. and diluted with Et₂O (50 mL). To this stirring solution was sequentially added distilled H₂O (0.5 mL), 1M NaOH (0.5 mL), and more distilled H₂O (1.2 mL). The reaction mixture was warmed to room temperature and stirred for 30 min. MgSO₄ (5 g) was added to the mixture, and the reaction stirred for another 30 min. The reaction mixture was filtered to remove the insoluble by-products, and the filtrate concentrated to provide compound 15 in 92% yield (1.726 g, 4.463 mmol) as a translucent grey solid. The allylic alcohol 15 was carried forward without further purification. R_{f}=0.20 (10% EtOAc in hexanes); ¹H NMR (300 MHz, CDCl₃) δ 5.60 (1H, t, J=7.0 Hz), 4.18-4.10 (2H, m), 4.06-3.96 (2H, m), 2.40-2.30 (2H, m), 2.18 (1H, dd, J=13.5, 3.0 Hz), 2.06 (1H, dd, J=12.0, 9.0 Hz), 1.87-1.78 (1H, m), 1.69-1.59 (1H, m), 1.43 (1H, br s), 0.89 (18H, s), 0.07 (6H, s), 0.06 (3H, s), 0.05 (3H, s); ¹³C NMR (75 MHz, CDCl₃) δ 138.4, 125.4, 68.3, 68.1, 58.5, 45.8, 43.6, 36.8, 26.1 (6C), 18.4 (2C), −4.5 (4C).

### [2-((3R,5R)-3,5-bis[tert-butyl(dimethyl)silyloxy]cyclohexylidene)ethyl](diphenyl)phosphine oxide (16).²¹

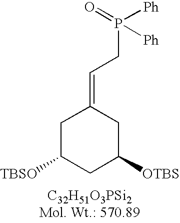

In a flame dried round bottom flask under argon atmosphere, a 2.15 M solution of n-BuLi in hexanes (1.47 mL, 3.16 mmol, 1.05 equiv) was added to a stirred solution of 15 (1.11 g, 3.01 mmol, 1 equiv) in THF (12 mL) at 0° C. To this mixture was added via cannula, a solution of freshly recrystallized p-toluenesulfonylchloride (602 mg, 3.16 mmol, 1.05 equiv) in THF (6 mL). The reaction was stirred at 0° C. for 2.5 hours. To this solution was added over a period of 30 min, a bright red solution of LiPPh₂, prepared separately in a separate flame dried flask under argon by adding a 2.15 M solution of n-BuLi in hexanes (1.54 mL, 3.31 mmol, 1.10 equiv) to a solution of HPPh₂ (0.575 mL, 3.31 mmol, 1.10 equiv) in THF (5 mL). The reaction mixture was allowed to stir at 0° C. for 1 h then warmed to room temperature. The reaction mixture was concentrated, and the residue dissolved in CHCl₃ (25 mL) and distilled H₂O (25 mL). To this mixture was added a 50% aqueous solution of H₂O₂ (1.73 mL, 30 mmol, 9.97 equiv), and the reaction mixture was stirred at room temperature for 3 h. The reaction was quenched with sat. NaHCO₃ (25 mL), the layers separated, and the aqueous layer extracted with CH₂Cl₂ (3×30 mL). The combined organic layers were extracted with brine (30 mL), then dried (MgSO₄), filtered, and concentrated in vacuo. The residue was purified by FCC on silica gel using a 1:1 ethyl acetate-hexanes mixture as eluent. The product was further recrystallized from diethyl ether to give 16 as a white solid in 75% yield (1.2888 g, 2.26 mmol). R_{f}=0.30 (30% EtOAc in hexanes); ¹H NMR (300 MHz, CDCl₃) δ 7.71-7.58 (4H, m), 7.46-7.33 (6H, m), 5.22 (1H, ddd, J=14.0, 7.0, 7.0 Hz), 3.91 (2H, m), 3.11 (1H, ddd, J=15.0, 8.0, 8.0 Hz), 2.99 (1H, ddd, J=15.0, 8.0, 8.0 Hz), 2.22-2.12 (1H, m), 2.00-1.79 (3H, m), 1.60 (2H, dd, J=5.0, 5.0 Hz), 0.80 (9H, s), 0.78 (9H, s), −0.04 (3H, s), −0.05 (6H, s), −0.06 (3H, s); ¹³C NMR (75 MHz, CDCl₃) δ 139.20 (d, J=12.0 Hz), 133.00 (d, J=98.0 Hz), 132.70 (d, J=98.0 Hz), 131.90 (2C), 131.30 (2C, d, J=9.5 Hz), 131.20 (2C, d, J=9.5 Hz), 128.70 (2C, d, J=11.5 Hz), 128.6 (2C, d, J=11.5 Hz), 113.90 (d, J=8.5 Hz), 68.0, 67.7, 45.3, 43.6, 37.4, 30.7 (d, J=70.0 Hz), 26.2 (3C), 26.1 (3C), 18.5, 18.4, −4.4 (4C).

### (1R,3aR,7aR)-1-[(1S)-2,2-dimethoxy-1-methylethyl]-7-methyl-octahydro-4H-inden-4-one (18)

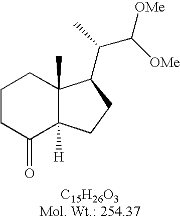

Ozone gas was bubbled through a solution of vitamin D₂ (17, ergocalciferol) (2.7071 g, 6.82 mmol, 1 equiv) in MeOH (72 mL) and CHCl₃ (8 mL) at −78° C. until a dark blue color persisted and then left for another hour. Argon was then bubbled through the reaction mixture until the solution turned clear. Me₂S (3.0 mL, 41 mmol, 6.0 equiv) was added to the reaction mixture at −78° C., and the reaction stirred for 1 hour, then warmed to room temperature and stirred for another 30 min. The conversion of the keto-aldehyde to the keto-acetal was carefully monitored by thin layer chromatography on silica gel (eluent: 1:4 ethyl acetate to hexanes). The reaction was immediately stopped upon appearance of a third spot indicating the epimerization of the C14 stereocenter. The reaction mixture was then concentrated and the crude loaded directly onto silica gel. Compound 18 was isolated via silica gel column chromatography (1:4 ethyl acetate to hexanes) in 65% yield (1.1313 g, 4.45 mmol) as a clear oil. R_{f}=0.40 (20% EtOAc in hexanes); ¹H NMR (400 MHz, CDCl₃) δ 4.09 (1H, d, J=2.5 Hz), 3.40 (3H, s), 3.35 (3H, s), 2.43 (1H, dd, J=11.0, 7.5 Hz), 2.28-2.15 (2H, m), 2.10-1.95 (2H, m), 1.92-1.82 (2H, m), 1.76-1.58 (4H, m), 1.57-1.47 (1H, m), 1.43-1.33 (1H, m), 0.95 (3H, d, J=6.5 Hz), 0.61 (3H, s); ¹³C NMR (100 MHz, CDCl₃) δ 211.6, 108.8, 61.6, 57.0, 56.1, 52.7, 50.1, 41.2, 39.4, 39.0, 27.2, 24.2, 19.4, 12.6, 12.1; IR (film) ν 2956, 1714, 1461, 1381, 1142, 1070, 959 cm⁻¹; LRMS (ESI): m/z (rel. intensity)=255 [38, (M+H)⁺], 223 (100), 191 (12), 107 (5), 74 (6); HRMS (ESI): m/z calcd. for [(M+H)⁺]=255.1955, found=255.1954.

### (1R,3R,7E,17β)-1,3-bis[tert-butyl(dimethyl)silyloxy]-17[(1S)-2,2-dimethoxy-1-methylethyl]-9,10-secoestra-5,7-diene (19)

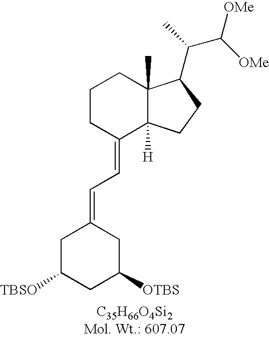

In a flame dried round bottom flask, cooled to −78° C. under argon, NaHMDS (2.574 mmol) was added to a solution of 16 (1.469 g, 2.574 mmol) in THF (30 mL). The reaction vessel was suspended above the ice bath for 5 min, then recooled to −78° C. A solution of 18 (0.6235 g, 2.451 mmol) in THF (10 mL) was cannulated into the reaction mixture over a period of 15 min. The reaction mixture was left to stir at −78° C. for 1 h, followed by warming to room temperature over a period of 30 min, and quenching with sat. NH₄Cl (25 mL). The layers were separated and the aqueous layer extracted with EtOAc (2×25 mL). The organic layers were combined and extracted with sat. NH₄Cl (2×25 mL), distilled H₂O (25 mL) and brine (25 mL), then dried (MgSO₄) and concentrated in vacuo to give the crude product. Compound 19 was isolated via FCC (20% EtOAc in hexanes) as a clear amorphous solid in 69% yield (1.027 g, 1.691 mmol). R_{f}=0.70 (10% EtOAc in hexanes); ¹H NMR (300 MHz, CDCl₃) δ 6.16 (1H, d, J=11.0 Hz), 5.82 (1H, d, J=11.0 Hz), 4.15 (1H, d, J=2.0 Hz), 4.13-4.02 (2H, m), 3.45 (3H, s), 3.39 (3H, s), 2.88-2.76 (1H, m), 2.46-2.33 (2H, m), 2.30-2.22 (1H, m), 2.16-1.50 (13H, m), 1.44-1.34 (2H, m), 0.98 (3H, d, J=6.5 Hz), 0.89 (9H, s), 0.88 (9H, s), 0.56 (3H, s), 0.07 (12H, m); ¹³C NMR (75 MHz, CDCl₃) δ 140.6, 133.8, 121.8, 116.3, 109.2, 68.3, 68.2, 57.2, 56.0, 55.9, 52.5, 46.2, 46.0, 44.0, 40.7, 40.1, 37.1, 29.0, 27.4, 26.1 (6C), 23.6, 22.6, 18.4 (2C), 12.3, 12.2, −4.3, −4.4, −4.5, −4.6; IR (film) ν 2951, 1739, 1619, 1471, 1361, 1254, 1187, 1089, 1026, 960, 921, 836 cm⁻¹; LRMS (EI): m/z (rel. intensity)=608 (10), 607 (20, M⁺), 592 (12), 590 (22), 576 (21), 575 (51), 574 (100), 534 (8), 533 (19), 518 (9), 458 (59), 442 (96), 239 (13), 237 (11); HRMS (EI): m/z calcd. for (M⁺)=606.4499, found=606.4490.

### (2S)-2-((1R,3R,7E,17β)-1,3-bis[tert-butyl(dimethyl)silyloxy]-9,10-secoestra-5,7-dien-17-yl)propanal (20)

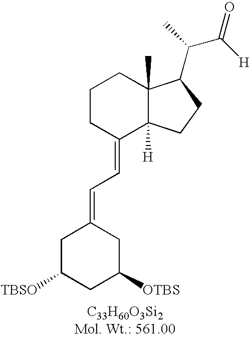

Trifluoroacetic acid (0.8 mL, 11 mmol, 24 equiv) was added to a vigorously stirred solution of 19 (273.0 mg, 0.450 mmol, 1 equiv) in CHCl₃ (4.8 mL) and distilled H₂O (2.4 mL) at 0° C. The mixture rapidly turned purple, then blue-green, then colorless. The reaction was monitored by thin layer chromatography on silica gel plates (eluent: 1:9 ethyl acetate to hexanes). After 25 minutes, the starting material spot completely converted to a new spot. The reaction was quenched with sat. NaHCO₃ (15 mL), the layers were separated and the aqueous layer extracted with EtOAc (2×15 mL). The organic layers were combined and extracted with sat. NaHCO₃ (2×15 mL), distilled H₂O (10 mL) and brine (10 mL), then dried (MgSO₄), filtered and concentrated in vacuo to give the crude product 20 as a clear oil in 89% yield (225.5 mg, 0.402 mmol). This product was carried forward without further purification. R_{f}=0.75 (10% EtOAc in hexanes); ¹H NMR (300 MHz, CDCl₃) δ 9.58 (1H, d, J=3.0 Hz), 6.16 (1H, d, J=11.0 Hz), 5.83 (1H, d, J=11.0 Hz), 4.17-4.02 (2H, m), 2.89-2.80 (1H, m), 2.46-2.22 (4H, m), 2.16-1.92 (4H, m), 1.84-1.54 (8H, m), 1.48-1.35 (2H, m), 1.14 (3H, d, J=6.5 Hz), 0.89 (9H, s), 0.87 (9H, s), 0.60 (3H, s), 0.06 (12H, m); ¹³C NMR (75 MHz, CDCl₃) δ 204.9, 139.7, 134.3, 121.6, 116.7, 68.3, 68.1, 55.7, 51.6, 50.0, 46.3 (2C), 43.9, 40.5, 37.0, 28.9, 26.8, 26.1 (6C), 23.5, 22.9, 18.4 (2C), 13.9, 12.8, −4.3, −4.4, −4.5, −4.6; IR (film) ν 2953, 2706, 1726, 1619, 1472, 1361, 1255, 1089, 1052, 1026, 1006, 960, 920, 836 cm⁻¹; LRMS (EI): m/z (rel. intensity)=560 (15, M⁺), 503 (20), 428 (75), 371 (20), 301 (30), 239 (25), 147 (35), 133 (45), 74 (100); HRMS (EI): m/z calcd. for (M⁺)=560.4081, found=560.4085.

### Ethyl (2E,4R)-4-((1R,3R,7E,17β)-1,3-bis[tert-butyl-(dimethyl)silyloxy]-9,10-secoestra-5,7-dien-17-yl)-2-methylpent-2-enoate (21)

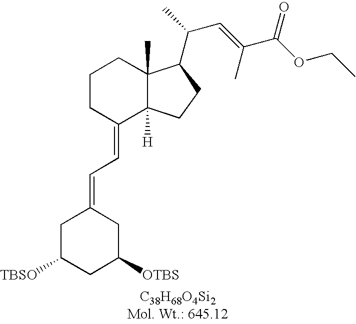

Ethyl 2-(triphenylphosphoranylidene) propanoate (0.3805 g, 1.050 mmol) was added to a solution of 20 (0.5610 g, 1.000 mmol) in toluene (10 mL) in a round bottom flask. The flask was fitted with a reflux condenser and the reaction mixture heated to reflux for 16 h by means of a heating mantle. The reaction mixture was concentrated, and the residue dissolved in hexanes to precipitate out the triphenylphosphine oxide by-product. The suspension was filtered and the filtrate concentrated and loaded directly onto silica gel. Compound 21 was isolated via FCC (10% EtOAc in hexanes) as a clear oil in 98% yield (0.6322 g, 0.98 mmol). R_{f}=0.60 (5% EtOAc in hexanes); ¹H NMR (300 MHz, CDCl₃) δ 6.58 (1H, dd, J=11.0 Hz), 6.15 (1H, d, J=11.0 Hz), 5.82 (1H, d, J=11.0 Hz), 4.19 (2H, q, J=7.0 Hz), 4.13-4.05 (2H, m), 2.88-2.79 (1H, m), 2.62-2.49 (1H, m), 2.43-2.30 (3H, m), 2.18-1.97 (3H, m), 1.88 (3H, d, J=1.5 Hz), 1.83-1.63 (6H, m), 1.60-1.50 (4H, m), 1.46-1.36 (1H, m), 1.31 (3H, t, J=7.0 Hz), 1.05 (3H, d, J=6.5 Hz), 0.90 (9H, s), 0.89 (9H, s), 0.62 (3H, s), 0.07 (12H, m); ¹³C NMR (75 MHz, CDCl₃) δ 168.7, 147.7, 140.2, 134.1, 125.0, 121.8, 116.6, 68.4, 68.3, 60.5, 56.6, 56.4, 46.2, 46.0, 44.1, 40.9, 37.3, 36.2, 29.0, 27.3, 26.2 (6C), 23.8, 22.6, 19.6, 18.4 (2C), 14.6, 13.0, 12.8, −4.3, −4.4 (2C), −4.5; IR (film) ν 2953, 2929, 2856, 1711, 1471, 1362, 1255, 1194, 1090, 1051, 1026, 960, 921, 835 cm⁻¹; LRMS (EI): m/z (rel. intensity)=644 (20, M⁺), 587 (20), 512 (40), 455 (10), 371 (10), 301 (15), 239 (35), 113 (45), 74 (100); HRMS (EI): m/z calcd. for (M⁺)=644.4656, found=644.4646.

### (2E,4R)-4-((1R,3R,7E,17β)-1,3-bis[tert-butyl-(dimethyl)silyloxy]-9,10-secoestra-5,7-dien-17-yl)-2-methylpent-2-en-1-ol (22)

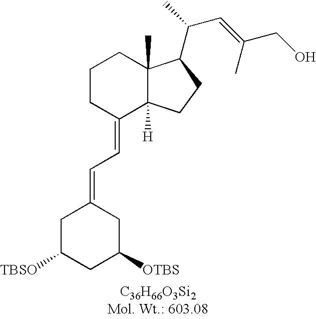

A 1.0 M solution of DIBAL-H in toluene (1.2 mL, 1.2 mmol, 3.0 equiv) was added to a solution of rigorously dried 21 (258.1 mg, 0.400 mmol, 1 equiv) in toluene (6 mL) at 0° C. The reaction was left to slowly warm to room temperature overnight. The reaction was then cooled to 0° C. and diluted with Et₂O (3.3 mL). To the stirring reaction was sequentially added distilled H₂O (0.040 mL), 1 M NaOH (0.040 mL), and more distilled H₂O (0.16 mL). The reaction was warmed to room temperature and stirred for 30 min. MgSO₄ was added to the mixture, and the reaction was stirred for another 30 min. The reaction was filtered to remove the insoluble by-products, and the filtrate concentrated. The crude product was purified by silica gel column chromatography using a gradient starting from 1:9 ethyl acetate to hexanes and ending with 1:4 ethyl acetate to hexanes, providing product 22 in 72% yield (172.5 mg, 0.29 mmol). R_{f}=0.30 (10% EtOAc in hexanes); ¹H NMR (400 MHz, CDCl₃) δ 6.17 (1H, d, J=11.0 Hz), 5.81 (1H, d, J=11.0 Hz), 5.21 (1H, d, J=10.0 Hz), 4.13-4.04 (2H, m), 3.99 (2H, s), 2.88-2.79 (1H, m), 2.43-2.34 (3H, m), 2.32-2.26 (1H, m), 2.11 (1H, dd, J=13.0, 8.0 Hz), 2.05-1.95 (2H, m), 1.83-1.63 (4H, m), 1.70 (3H, s), 1.59-1.47 (3H, m), 1.42-1.12 (5H, m), 0.99 (3H, d, J=6.5 Hz), 0.89 (9H, s), 0.88 (9H, s), 0.60 (3H, s), 0.07 (12H, m); ¹³C NMR (100 MHz, CDCl₃) δ 140.6, 133.8, 133.3, 131.5, 121.8, 116.3, 69.5, 68.3, 68.1, 57.0, 56.5, 46.2, 45.8, 43.9, 40.8, 37.1, 35.3, 29.0, 27.7, 26.2 (6C), 23.7, 22.5, 20.8, 18.4 (2C), 14.4, 12.7, −4.3, −4.4 (2C), −4.5; IR (film) ν 3348, 2952, 1619, 1471, 1361, 1255, 1090, 1025, 961, 906, 836 cm⁻¹; LRMS (EI): m/z (rel. intensity)=602 (25, M⁺), 470 (25), 371 (10), 301 (25), 237 (30), 143 (30), 74 (100); HRMS (EI): m/z calcd. for (M⁺)=602.4550, found=602.4542.

### (2E,4R)-4-((1R,3R,7E,17β)-1,3-bis[tert-butyl-(dimethyl)silyloxy]-9,10-secoestra-5,7-dien-17-yl)-2-methylpent-2-enal (23)

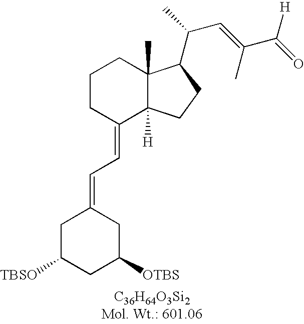

Dess-Martin periodinane (0.4988 g, 1.176 mmol) was added to a stirring solution of 22 (0.5674 g, 0.9408 mmol) in CH₂Cl₂ (10 mL). The reaction mixture was stirred for 1 h at room temperature, then diluted with Et₂O (20 mL) and quenched with sat. NaHCO₃ (40 mL) and sat. Na₂S₂O₃ (10 mL). The reaction mixture was stirred until the milky white organic layer became clear (approx. 1 h). The layers were separated and the aqueous layer extracted with Et₂O (2×25 mL). The organic layers were combined and extracted with distilled H₂O (25 mL) and brine (25 mL), then dried (MgSO₄), and concentrated in vacuo to give the crude product. Compound 23 was isolated via FCC (10% EtOAc in hexanes) as a translucent amorphous solid in 86% yield (0.4863 g, 0.8091 mmol). R_{f}=0.70 (10% EtOAc in hexanes); ¹H NMR (400 MHz, CDCl₃) δ 9.37 (1H, s), 6.29 (1H, d, J=10.5 Hz), 6.16 (1H, d, J=11.0 Hz), 5.82 (1H, d, J=11.0 Hz), 4.15-4.05 (2H, m), 2.88-2.81 (1H, m), 2.79-2.70 (1H, m), 2.42-2.34 (2H, m), 2.30-2.24 (1H, m), 2.16-1.98 (3H, m), 1.78 (3H, s), 1.76-1.51 (9H, m), 1.46-1.20 (2H, m), 1.10 (3H, d, J=6.5 Hz), 0.89 (9H, s), 0.88 (9H, s), 0.62 (3H, s), 0.06 (12H, m); ¹³C NMR (75 MHz, CDCl₃) δ 195.9, 160.3, 139.9, 136.5, 134.2, 121.7, 116.6, 68.2, 68.1, 56.2, 56.1, 46.2, 46.1, 43.9, 40.7, 37.1, 36.6, 28.9, 27.3, 26.1 (6C), 23.6, 22.5, 19.4, 18.4 (2C), 12.7, 9.9, −4.3, −4.4, −4.5, −4.6; IR (film) ν 2952, 2956, 1690, 1469, 1253, 1086, 1051, 1024, 959, 920, 834 cm⁻¹; LRMS (EI): m/z (rel. intensity)=600 (5, M⁺), 468 (20), 301 (5), 277 (100), 201 (20), 199 (20), 183 (20), 149 (20), 77 (45); HRMS (EI): m/z calcd. for (M⁺)=600.4394, found=600.4387.

### Methyl-(2E,4E,6R)-6-((1R,3R,7E,17β)-1,3-bis-[tert-butyl(dimethyl)silyloxy]-9,10-secoestra-5,7-dien-17-yl)-4-methylhepta-2,4-dienoate (24)

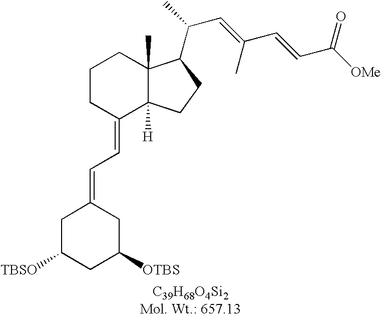

Methyl (triphenylphosphoranylidene)acetate (0.2840 g, 0.8496 mmol) was added to a solution of 23 (0.4863 g, 0.8091 mmol) in toluene (8 mL) in a round bottom flask. The flask was fitted with a reflux condenser, and the reaction mixture heated to reflux for 16 h by means of a heating mantle. The reaction mixture was concentrated, and the residue dissolved in hexanes to precipitate out the triphenylphosphine oxide by-product. The suspension was then filtered, and the filtrate concentrated and loaded directly onto silica gel. Compound 24 was isolated via FCC (10% EtOAc in hexanes) as a clear oil in 95% yield (0.5051 g, 0.7686 mmol). R_{f}=0.70 (10% EtOAc in hexanes); ¹H NMR (400 MHz, CDCl₃) δ 7.30 (1H, d, J=16.0 Hz), 6.16 (1H, d, J=11.0 Hz), 5.80 (1H, d, J=11.0 Hz), 5.78 (1H, d, J=16.0 Hz), 5.71 (1H, d, J=10.0 Hz), 4.12-4.03 (2H, m), 3.75 (3H, s), 2.86-2.78 (1H, m), 2.62-2.52 (1H, m), 2.40-2.33 (2H, m), 2.30-2.24 (1H, m), 2.11 (1H, dd, J=13.0, 8.0 Hz), 2.06-1.96 (2H, m), 1.80 (3H, s), 1.75-1.62 (5H, m), 1.58-1.30 (5H, m), 1.20-1.10 (1H, m), 1.02 (3H, d, J=6.5 Hz), 0.88 (9H, s), 0.87 (9H, s), 0.59 (3H, s), 0.06 (12H, m); ¹³C NMR (100 MHz, CDCl₃) δ 168.0, 150.5, 148.7, 140.3, 133.9, 129.8, 121.7, 116.4, 115.1, 68.3, 68.1, 56.6, 56.3, 51.7, 46.2, 45.9, 43.9, 40.7, 37.1, 36.3, 28.9, 27.5, 26.1 (6C), 23.6, 22.5, 20.1, 18.4 (2C), 12.9, 12.7, −4.3, −4.4 (2C), −4.5; IR (film) ν 2952, 2856, 1721, 1622, 1435, 1361, 1312, 1254, 1169, 1088, 1024, 960, 919, 835 cm⁻¹; LRMS (EI): m/z (rel. intensity)=656 (10, M⁺), 599 (15), 524 (25), 301 (20), 237 (25), 125 (30), 93 (85), 74 (100); HRMS (EI): m/z calcd. for (M⁺)=656.4656, found=656.4645.

### (2E,4E,6R)-6-((1R,3R,7E,17β)-1,3-bis[tert-butyl-(dimethyl)silyloxy]-9,10-secoestra-5,7-dien-17-yl)-4-methylhepta-2,4-dienoic acid (25)

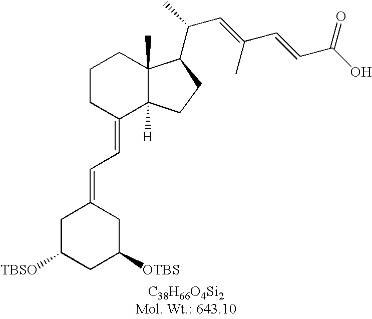

LiOH.H₂O (11.8 mg, 0.281 mmol, 7.47 equiv) was added to a stirring solution of 24 (24.7 mg, 0.0376 mmol, 1 equiv) in THF (1 mL), MeOH (0.4 mL) and H₂O (0.4 mL). The reaction vessel was fitted with a reflux condenser, and the reaction brought to reflux for 2.5 h. The reaction was cooled to room temperature and diluted with EtOAc (10 mL), then quenched with a pH 1 solution of KHSO₄ (10 mL). The layers were separated and the aqueous layer further extracted with EtOAc (5 mL). The organic layers were combined and extracted with brine (5 mL), then dried (MgSO₄), and concentrated in vacuo to give the crude product 25. This product was carried forward without further purification. If desired, product 25 can be purified by FCC (1:1 ethyl acetate to hexanes). R_{f}=0.30 (20% EtOAc in hexanes); ¹H NMR (300 MHz, CDCl₃) δ 10.00-9.30 (1H, br s), 7.39 (1H, d, J=15.5 Hz), 6.16 (1H, d, J=11.0 Hz), 5.82 (2H, m), 5.78 (1H, d, J=15.5 Hz), 4.15-4.00 (2H, m), 2.87-2.78 (1H, m), 2.63-2.53 (1H, m), 2.44-2.24 (3H, m), 2.16-1.96 (3H, m), 1.83 (3H, s), 1.82-1.36 (11H, m), 1.04 (3H, d, J=6.5 Hz), 0.89 (9H, s), 0.88 (9H, s), 0.60 (3H, s), 0.06 (12H, m); ¹³C NMR (75 MHz, CDCl₃) δ 173.1, 152.7, 149.8, 140.3, 134.0, 129.9, 121.7, 116.5, 114.8, 68.3, 68.1, 56.6, 56.3, 46.2, 46.0, 43.9, 40.7, 37.1, 36.4, 28.9, 27.5, 26.1 (6C), 23.6, 22.5, 20.1, 18.4 (2C), 12.9, 12.7, −4.3, −4.4, −4.5, −4.6; IR (film) ν 3000 (br), 2956, 1686, 1618, 1417, 1254, 1207, 1088, 1026, 908, 834, 801 cm⁻¹; LRMS (ESI): m/z (rel. intensity)=681 [6, (M+K)⁺], 665 [79, (M+Na)⁺], 643 (11, M⁺), 641 (20), 519 (18), 512 (27), 511 (100), 510 (11), 509 (39), 497 (11), 397 (41), 381 (13), 380 (16), 379 (64); HRMS (ESI): m/z calcd. for [(M+H)⁺]=643.4572, found=643.4572.

### (2E,4E,6R)-6-[(1R,3R,7E,17β)-1,3-dihydroxy-9,10secoestra-5,7-dien-17-yl]-N-hydroxy-4-methylhepta-2,4-dienamide (6)

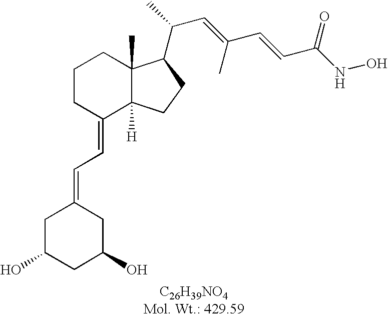

Oxalyl chloride (5.0 μl, 0.059 mmol, 1.57 equiv) was added to a solution of the rigorously dried crude product 25 (approximately 0.0376 mmol, 1 equiv) and N,N-dimethylformamide (0.6 μl, 7.7 micromole, 0.2 equiv) in dry dichloromethane (1 mL) at 0° C. The reaction mixture rapidly turned yellow and was left stirring at 0° C. for 90 minutes, at which time N,N-diisopropylethylamine (21 μl, 0.12 mmol, 3.2 equiv) was added followed by a solution of O-(tert-butyldimethylsilyl)hydroxylamine (11.9 mg, 0.081 mmol, 2.15 equiv) in dry dichloromethane (0.235 mL). The reaction was left to stir at 0° C. for 2 hours and then at room temperature for an additional 2 hours. The reaction was quenched by diluting with ethyl acetate (10 mL) and a 1M citric acid aqueous solution (10 mL). The layers were separated and the aqueous layer further extracted with ethyl acetate (5 mL). The combined organic layers were extracted with distilled water (5 mL) and brine (5 mL, then dried (MgSO₄), filtered and concentrated in vacuo. This crude product was dissolved in CDCl₃ (0.5 mL) and CD₃CN (0.5 mL) and then placed in a plastic vial. To this solution was added a 48 wt. % HF aqueous solution (50 μl) followed by an additional 50 μl after 2.5 hours (total 2.8 mmol, 73 equiv). The reaction was monitored by ¹H NMR and TLC and was complete after 4 hours. The reaction was quenched by diluting with ethyl acetate (10 mL) and a 1M citric acid aqueous solution (10 mL). The layers were separated and the aqueous layer was further extrated with ethyl acetate (5 mL). The combined organic layers were washed with distilled water (5 mL) and brine (5 mL), then dried with MgSO₄, filtered and evaporated in vacuo. The crude product was purified by means of octadecyl-functionalized reverse phase silica gel column chromatography using a solvent gradient starting from distilled water with 0.05% trifluoroacetic acid and ending with pure methanol. The product 6 was isolated as a white amorphous solid in 41% yield (6.6 mg, 0.015 mg) from the methyl ester 24. R_{f}=0.30 [(88:10:2) CH₂Cl₂:MeOH:CH₃COOH]; ¹H NMR (300 MHz, CD₃OD) δ 7.17 (1H, d, J=13.0 Hz), 6.20 (1H, d, J=10.5 Hz), 5.93-5.66 (3H, m), 4.08-3.94 (2H, m), 2.88-2.80 (1H, m), 2.64-2.55 (2H, m), 2.44-2.36 (1H, m), 2.24-2.11 (2H, m), 2.08-1.95 (2H, m), 1.99 (3H, s), 1.79 (3H, s), 1.70-1.35 (8H, m), (3H, d, J=6.0 Hz), 0.63 (3H, s), 4 exchangeable protons unobserved; ¹³C NMR (75 MHz, CD₃OD) δ 166.8, 147.9, 146.9, 141.6, 133.9, 130.9, 123.2, 117.1, 115.5, 67.9, 67.6, 57.9, 57.3, 46.9, 45.4, 42.7, 41.7, 37.6, 37.1, 29.8, 28.2, 24.5, 23.3, 20.5, 13.2, 12.9; IR (film) ν 3221 (br), 2929, 2869, 1645, 1611, 1446, 1377, 1043, 976 cm⁻¹; LRMS (ESI): m/z (rel. intensity)=859 [9, (2M+H)⁺], 452 [17, (M+Na)⁺], 430 [100, (M+H)⁺], 412 (8), 397 (10), 390 (8); HRMS (ESI): m/z calcd. for [(M+H)⁺]=430.2952, found=430.2952.

Lythgoe-Inhoffen Diol (27).

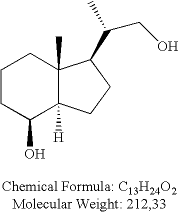

A flame-dried 100 mL three-necked flask was charged sequentially with 28 mg (0.33 mmol) of NaHCO₃, 20 mL of anhydrous MeOH, 60 mL of anhydrous CH₂Cl₂, and 2.0 g (5.12 mmol) of ergocalciferol (17). The solution was cooled to −78° C. and treated with O₃ until a deep blue color developed and persisted. The solution was subsequently flushed with Argon for 10-15 min until the blue color faded. Solid sodium borohydride (1.68 g, 44.55 mmol) was added portionwise over a period of 10 min at −78° C. until complete disappearance of starting material was observed by TLC. The reaction mixture was warmed to 0° C. and stirred for 3 h. After being stirred for an additional 30 min at rt., the mixture was quenched with 1 N HCl, extracted with EtOAc (3×50 mL), dried over MgSO₄, filtered, and concentrated in vacuo. Purification by silica gel chromatography (30% EtOAc in hexanes) afforded 670 mg (3.17 mmol) of Lythgoe-Inhoffen Diol 27 in 62% yield as a white solid. R_{f}=0.5 (50% EtOAc in hexanes); Mp 108-110° C. (lit. Mp 109-110° C.)³²; ¹H NMR (400 MHz, CDCl₃) δ 4.08 (1H, br s), 3.63 (1H, dd, J=10.4, 2.8 Hz), 3.37 (1H, J=10.0, 6.8 Hz), 1.98 (1H, d, J=12.8 Hz), 1.90-1.75 (3H, m), 1.60-1.40 (5H, m), 1.38-1.29 (4H, m), 1.22-1.13 (2H, m), 1.02 (3H, d, J=6.8 Hz), 0.95 (3H, s); ¹³C NMR (400 MHz, CDCl₃) δ 69.1, 67.7, 52.9, 52.3, 41.8, 40.2, 38.2, 33.5, 26.6, 22.5, 17.4, 16.6, 13.5; IR (KBr) ν 3621, 3464, 3017, 2943 cm⁻¹.

### (S)-2-((1R,3aR,4S,7aR)-4-(tert-butyldimethylsilyloxy)-7a-methyloctahydro-1H-inden-1-yl)propan-1-ol (28)

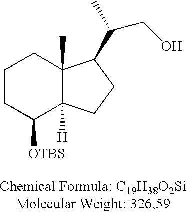

To a solution of Lythgoe-Inhoffen diol 27 (2.017 g, 9.5 mmol) in 60 mL of dry DMF under Argon was added 5.71 g (38 mmol) of tert-butyldimethylsilylchloride, followed by 5.54 mL (42.7 mmol) of NEt₃ and 5.69 g (38 mmol) of sodium iodide. The reaction mixture was then refluxed for 30 min. The reaction was quenched with H₂O (10 mL) and concentrated in vacuo. The residue was dissolved in EtOAc (100 mL) and washed with H₂O (2×30 mL), the aqueous portion was extracted with EtOAc (30 mL) and the combined organic layers were washed with brine (30 mL), dried over MgSO₄, filtered, concentrated by rotary evaporation, and immediately purified by silica gel chromatography (10% EtOAc in hexanes) to afford 4.013 mg (9.12 mmol) of bis-silylated diol intermediate in 96% yield. In a flame-dried round bottom flask under argon, 4.013 mg (9.12 mmol) of the bis-silylated diol was dissolved in 80 mL of anhydrous THF and 4 mL of NEt₃. To this stirring solution was added 10.9 mL of TBAF (10.9 mmol, 1 M solution in THF). The resulting reaction mixture was stirred at rt. for 3 hours, concentrated by rotary evaporation, and purified by silica gel column chromatography (20% EtOAc in hexanes) to afford 2.916 g (8.94 mmol) of the desired alcohol 28 in 98% yield. R_{f}=0.5 (20% EtOAc in hexanes); ¹H NMR (400 MHz, CDCl₃) δ 4.08 (1H, br s), 3.57 (1H, dd, J=9.8, 3.0 Hz), 3.26 (1H, dd, J=9.0, 7.8 Hz), 1.99 (1H, d, J=13.6 Hz), 1.87-1.75 (3H, m), 1.58-1.40 (5H, m), 1.35-1.28 (3H, m), 1.40-1.09 (2H, m), 0.97 (3H, d, J=6.8), 0.94 (3H, s), 0.89 (9H, s), 0.025 (6H, s); ¹³C NMR (400 MHz, CDCl₃) δ 69.3, 67.7, 53.2, 52.3, 41.8, 40.2, 38.5, 33.6, 26.6, 26.0, 22.6, 18.4, 17.4, 16.8, 13.6, −5.3, −5.4; IR (KBr) ν 3300, 1470, 1250, 1160 cm⁻¹.

### (S)-2-((1R,3aR,4S,7aR)-4-(tert-butyldimethylsilyloxy)-7a-methyloctahydro-1H-inden-1-yl)-1-iodopropane (29)

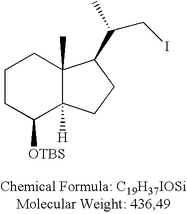

To an ice cooled solution of PPh₃ (4.82 g, 18.4 mmol) and imidazole (3.26 g, 48.0 mmol) in 300 mL of CH₂Cl₂ was added portionwise 9.34 g of 12 (36.8 mmol). The cooled mixture, which became heterogeneous after 5 min, was stirred for 35 min and treated with a solution of the primary alcohol 28 (2.61 g, 8 mmol) in 100 mL of CH₂Cl₂ over a period of 30 min. The reaction mixture was then stirred at rt. for 4 h. The reaction was quenched with a solution of Na₂SO₄ (30 mL, 2.5%). The organic layer was washed with H₂O (30 mL), brine (30 mL), dried over MgSO₄, concentrated in vacuo, and then purified by silica gel column chromatography (5% EtOAc in hexanes) to afford 3.14 g (6.96 mmol) of iodide 29 in 87% yield as a white solid. R_{f}=0.6 (5% EtOAc in hexanes); Mp 40-41° C. (lit. Mp 41-42° C.)³³; ¹H NMR (400 MHz, CDCl₃) δ 3.99 (1H, s), 3.32 (1H, d, J=9.6 Hz), 3.17 (1H, dd, J=9.6, 5.2 Hz), 1.90 (1H, d, J=12.8 Hz), 1.84-1.76 (2H, m), 1.66 (1H, d, J=13.6 Hz), 1.59-1.54 (1H, m), 1.40-1.08 (8H, m), 0.98 (3H, d, J=5.2 Hz), 0.94 (3H, s), 0.88 (9H, s), −0.01 (6H, s); ¹³C NMR (400 MHz, CDCl₃) δ 69.3, 56.0, 52.7, 42.1, 40.3, 36.4, 34.3, 26.6, 25.8, 22.9, 21.7, 20.7, 18.0, 17.6, 14.6, −4.8, −5.2; IR (KBr), ν 2932, 2857, 1462, 1375, 1253, 1160, 1084, 1032, 836, 774 cm⁻¹.

### (S)-5-((1R,3aR,4S,7aR)-4-(tert-butyldimethylsilyloxy)-7a-methyloctahydro-1H-inden-1-yl)-1-ethoyhex-1-ene (30)

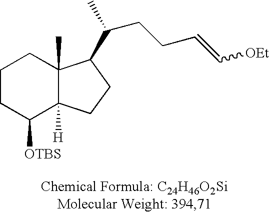

In a flame-dried round bottom flask under argon, iodide 29 (678 mg, 1.5 mmol) was dissolved in 3 mL of dry Et₂O, the solution cooled to −78° C. and 1.42 mL of t-BuLi (3.15 mmol, 2.22 M solution in pentane) was slowly added (1 h). The solution was stirred at −78° C. for 1 h, then warmed to 0° C. for 5 min and then recooled to −78° C. A solution of acrolein acetal (251 μL, 1.65 mmol) in dry Et₂O (2 mL) was slowly added and the mixture warmed to rt. Stirring was continued for an additional 30 min followed by quenching with a saturated solution of NH₄Cl (10 mL). Extraction with Et₂O (3×20 mL) afforded an organic phase that was washed with H₂O (20 mL), brine (20 mL) and dried over MgSO₄, concentrated in vacuo, and then purified by silica gel column chromatography (hexanes then 1% Et₂O in hexanes) to afford 444 mg (1.12 mmol) of the enol ether 30 as a mixture of E/Z isomers (86/14) in 75% yield. R_{f}=0.2 (hexanes); ¹H NMR (400 MHz, CDCl₃) 6 (30 E): 6.21 (1H, d, J=12.4 Hz), 4.74 (1H, dt, J=12.4, 6.2 Hz), 3.98 (1H, s), 3.69 (2H, q, J=6.8 Hz), 1.95 (2H, br d, J=12.4 Hz), 1.81-1.73 (3H, m), 1.65 (1H, br d, J=13.2 Hz), 1.60-1.51 (1H, m), 1.42-1.29 (10H, m), 1.27-1.00 (3H, m), 0.95-0.80 (15H, m), 0.00 (3H, s), −0.01 (3H, m); (30 E): 5.90 (1H, d, J=9.2 Hz), 4.30 (1H, dd, J=9.2, 7.5 Hz), 3.98 (1H, br s), 3.77 (2H, q, J=7.5 Hz), 1.95 (2H, br d, J=12.4 Hz), 1.81-1.73 (3H, m), 1.65 (1H, br d, J=13.2 Hz), 1.60-1.51 (1H, m), 1.42-1.29 (10H, m), 1.27-1.00 (3H, m), 0.95-0.80 (15H, m), 0.00 (3H, s), −0.01 (3H, m); ¹³C NMR (400 MHz, CDCl₃) δ 145.6, 104.8, 69.5, 64.5, 56.7, 53.0, 42.1, 40.7, 37.0, 34.8, 34.4, 27.3, 25.8, 24.5, 23.0, 18.5, 18.0, 17.7, 14.8, 13.7, −4.8, −5.2; IR (KBr) ν 2931, 2856, 1652, 1469, 1374, 1251, 1165, 1083, 1023, 977, 924, 836, 774 cm⁻¹; HRMS (ESI): m/z calcd. for [(M+Na)⁺]=417.3165, found=417.3155.

### (1R,3aR,4S,7aR)-1-((R)-5-(1,3-dioxolan-2-yl)pentan-2-yl)-7a-methyl-octahydro-1H-inden-4-ol (31)

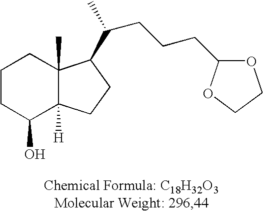

To a solution of enol ether 30 (524 mg, 1.32 mmol) in 10 mL of CH₃CN and 10 mL of CH₂Cl₂, HF (1 mL of a 48% solution in H₂O) was added and the solution was stirred at rt. overnight. The reaction was quenched by the addition of a saturated solution of NaHCO₃ until no further effervescence was observed. The solution was extracted with CH₂Cl₂ (3×30 mL), the combined organic layers were washed with H₂O (20 mL), brine (20 mL), dried over MgSO₄ and then concentrated in vacuo. The crude oil was dissolved in toluene (30 mL), then ethylene glycol (734 μL, 13.2 mmol) and a catalytic quantity of PTSA were added and the solution was refluxed in a Dean Stark apparatus for 30 min. The solution was concentrated in vacuo and then purified by silica gel column chromatography (25% EtOAc in hexanes) to afford 335 mg (1.13 mmol) of the acetal 31 in 86% yield. R_{f}=0.24 (25% EtOAc in hexanes); ¹H NMR (400 MHz, CDCl₃) δ 4.83 (1H, t, J=4.6 Hz), 4.06 (1H, s), 3.99-3.94 (2H, m), 3.88-3.81 (2H, m), 1.98 (1H, d, J=12.8 Hz), 1.86-1.77 (3H, m), 1.65-1.20 (13H, m), 1.19-1.01 (3H, m), 0.91 (3H, s), 0.89 (3H, d, J=7.2 Hz); ¹³C NMR (400 MHz, CDCl₃) δ 104.7, 69.4, 64.8, 56.4, 52.6, 41.8, 40.3, 35.6, 35.2, 34.4, 35.5, 27.1, 22.5, 20.5, 18.4, 17.4, 13.5; IR (KBr) ν 3505 (br), 2941, 2869, 1458, 1408, 1373, 1141, 1031, 990, 941 cm⁻¹; HRMS (ESI): m/z calcd. for [(M+Na)⁺]=319.3349, found=319.2239.

### (1R,3aR,7aR)-1-((R)-(dioxolan-2-yl)pentan-2-yl)-7a-methyloctahydroinden-4-one (32)

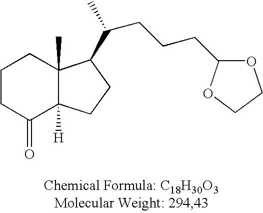

To a solution of acetal 31 (326 mg, 1.10 mmol) in dry CH₂Cl₂ and comprising celite (400 mg), was added 474 mg of PCC (2.2 mmol). The reaction mixture was stirred at rt. for 2 h, then filtered, concentrated in vacuo and purified by silica gel column chromatography (50% EtOAc in hexanes) to afford 307 mg (1.05 mmol) of the acetal 32 in 86% yield. R_{f}=0.6 (50% EtOAc in hexanes); ¹H NMR (400 MHz, CDCl₃) δ 4.82 (1H, t, J=4.8 Hz), 3.98-3.90 (2H, m), 3.89-3.80 (2H, m), 2.42 (1H, dd, J=11.6, 7.6 Hz), 2.30-2.14 (2H, m), 2.14-2.04 (1H, m), 2.02-1.80 (3H, m), 1.78-1.22 (11H, m), 1.18-1.04 (1H, m), 0.94 (3H, d, J=6.4 Hz), 0.61 (3H, s); ¹³C NMR (400 MHz, CDCl₃) δ 212.1, 104.6, 64.8, 61.9, 56.4, 49.9, 40.9, 38.9, 35.6, 35.4, 34.3, 27.5, 24.0, 20.4, 19.0, 18.6, 12.4; IR (KBr) ν 2952, 2874, 1712, 1459, 1378, 1231, 1141, 1033, 943 cm⁻¹; HRMS (ESI): m/z calcd. for [(M+Na)⁺]=317.2093, found=317.2082.

### (1R,3R,7E,17β)-1,3-bis{[tert_butyl(dimethyl)silyl]oxy}-17-[4(1,3-dioxolan-2-yl)-1-methylbutyl]-9,10-secogona-5,7-diene (33)

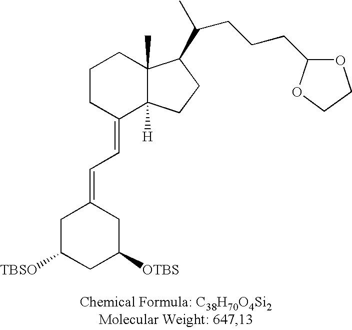

In a flame dried round bottom flask, cooled to −78° C. under argon, NaHMDS (0.430 mmol, 1 M solution in THF) was added to a solution of phosphine oxide 16 (249 mg, 0.430 mmol) in THF (5 mL). The reaction vessel was suspended above the ice bath for 5 min, then recooled to −78° C. A solution of 32 (118 mg, 0.415 mmol) in THF (1 mL) was cannulated into the reaction mixture over a period of 5 min. The reaction was left to stir at −78° C. for 1 h then warmed to room temperature for 1 h, and quenched with sat. NH₄Cl (25 mL). The layers were separated and the aqueous layer extracted with EtOAc (2×25 mL). The organic layers were combined and extracted with sat. NH₄Cl (2×25 mL), H₂O (25 mL) and brine (25 mL), dried (MgSO₄), and concentrated in vacuo. The crude product was purified by silica gel column chromatography (20% EtOAc in hexanes) to afford 203 mg (0.31 mmol) of 33 in 75% yield. R_{f}=0.6 (20% EtOAc in hexanes); ¹H NMR (400 MHz, CDCl₃) δ 6.16 (1H, d, J=11.2 Hz), 5.81 (1H, d, J=11.2 Hz), 4.84 (1H, t, J=9.0 Hz), 4.13-4.01 (2H, m), 4.00-3.92 (2H, m), 3.91-3.81 (2H, m), 2.80 (1H, d, J=12.4 Hz), 2.42-2.32 (2H, m), 2.30-2.21 (1H, m), 2.14-2.05 (1H, m), 2.02-1.94 (2H, m), 1.93-1.73 (2H, m), 1.70-1.18 (15H, m), 1.16-1.02 (1H, m), 0.93 (3H, d, J=6.4 Hz), 0.87 (9H, s), 0.86 (9H, s), 0.52 (3H, s), 0.04 (12H, s); ¹³C NMR (400 MHz, CDCl₃) δ 140.8, 133.6, 121.7, 116.1, 104.7, 68.1, 67.9, 68.1, 56.3, 56.2, 46.0, 45.6, 43.7, 40.6, 36.7, 36.0, 35.8, 34.4, 28.7, 27.7, 25.9, 25.8, 23.4, 22.2, 20.6, 18.7, 18.1, 12.0, −4.6, −4.7, −4.8, −4.9; IR (KBr) ν 2950, 2857, 1468, 1253, 1127, 1087, 836, 775 cm⁻¹; HRMS (ESI): m/z calcd. for [(M+Na)⁺]=669.4711, found=669.4691.

### (5S)-5-((1R,3R,7E,17β)-1,3-bis[tert-butyl(dimethyl)silyl-oxy]-9,10-secoestra-5,7-dien-17-yl)hexanal (34)

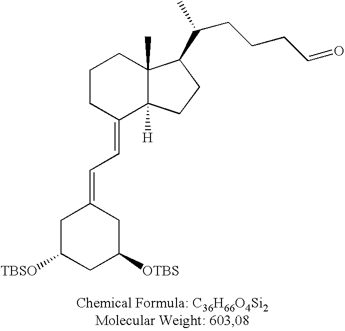

A solution of 33 (360 mg, 0.556 mmol) and PPTS (140 mg, 0.556 mmol) in acetone (10 mL) and H₂O (0.2 mL) was refluxed overnight. The solvent was removed in vacuo, Et₂O (30 mL) was added, the mixture washed with sat. NaHCO₃ (10 mL), brine (10 mL), dried over MgSO₄ and concentrated in vacuo. In a flame dried round bottom flask under argon, the crude material was dissolved into dry THF (8 mL) and cooled to −78° C. 2,4,6-collidine (367 μL, 2.78 mmol) and then TBSOTf (208 μL, 1.22 mmol) were then added. The cold bath was removed and the reaction warmed to rt. and stirred for an additional 20 min. The reaction was quenched with H₂O (10 mL), extracted with CH₂Cl₂ (3×20 mL), dried over MgSO₄ and concentrated in vacuo and then purified by silica gel column chromatography (CH₂Cl₂) to afford 261 mg (0.43 mmol) of aldehyde 34 in 78% yield. R_{f}=0.6 (CH₂Cl₂); ¹H NMR (400 MHz, CDCl₃) δ 9.76 (1H, s), 6.16 (1H, d, J=12.0 Hz), 5.81 (1H, d, J=12.0 Hz), 4.40-4.43 (2H, m), 2.80 (1H, dd, J=11.0, 3.1 Hz), 2.43-2.34 (4H, m), 2.24 (1H, br d, J=13.6 Hz), 2.89 (1H, dd, J=12.8, 8.4 Hz), 2.05-1.85 (2H, m), 1.85-1.20 (15H, m), 1.20-1.05 (1H, m), 0.94 (3H, d, J=6.0 Hz), 0.87 (9H, s), 0.86 (9H, s), 0.53 (3H, s), 0.05 (12H, s); ¹³C NMR (400 MHz, CDCl₃) δ 202.9, 140.6, 133.7, 121.7, 116.2, 68.1, 67.9, 56.22, 56.20, 46.0, 45.6, 44.3, 43.7, 40.5, 36.7, 36.0, 35.4, 28.7, 27.7, 25.87, 25.84, 23.4, 22.2, 18.7, 18.15, 18.10, 12.0, −4.6, −4.7, −4.8, −4.9; IR (KBr) ν 2950, 2883, 2856, 1728, 1468, 1377, 1361, 1253, 1087, 1025, 836, 775 cm⁻¹; HRMS (ESI): m/z calcd. for [(M+Na)⁺]=625.4448, found=625.4434.

### (5S)-5-((1R,3R,7E,17β)-1,3-bis[tert-butyl(dimethyl)silyl-oxy]-9,10-secoestra-5,7-dien-17-yl)-hexanoic acid (35)

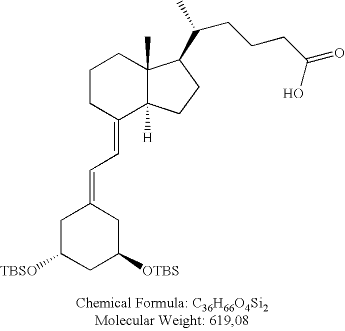

To a stirred solution of aldehyde 34 (72 mg, 0.12 mmol) and 2-methyl-2-butene (620 μL, 1.2 mmol) in t-BuOH (2 mL), was added a freshly prepared solution of NaClO₂ (33 mg, 0.36 mmol) and NaH₂PO₄ (83 mg, 0.60 mmol) in H₂O (2 mL). The reaction mixture was vigorously stirred at rt. for 1 h. H₂O (15 mL) was then added and the reaction mixture extracted with Et₂O (3×20 mL). The combined organic layers were washed with brine (10 mL) and dried over MgSO₄. The solution was concentrated in vacuo and then purified by silica gel column chromatography (10% to 40% EtOAc in hexanes) to afford 68 mg (0.11 mmol) of acid 35 in 91% yield. R_{f}=0.6 (40% EtOAc in hexanes); ¹H NMR (400 MHz, CDCl₃) δ 6.16 (1H, d, J=10.8 Hz), 5.81 (1H, d, J=10.8 Hz), 4.15-4.01 (2H, m), 2.81 (1H, br d, J=11.6 Hz), 2.42-2.21 (5H, m), 2.12-1.05 (14H, m), 0.94 (3H, d, J=7.2 Hz), 0.87 (9H, s), 0.86 (9H, s), 0.53 (3H, s), 0.04 (12H, s); ¹³C NMR (400 MHz, CDCl₃) δ 180.0, 140.7, 133.7, 121.7, 116.1, 68.1, 68.0, 56.2, 46.0, 45.6, 43.7, 40.5, 36.7, 35.9, 35.3, 34.5, 28.7, 27.7, 25.9, 25.8, 23.4, 22.2, 21.3, 18.7, 18.2, 18.1, 12.0, −4.6, −4.7, −4.8, −4.9; IR (KBr) ν 3435 (br), 2951, 1637, 1459, 1247, 1086, 835, 780, 668 cm⁻¹; HRMS (ESI): m/z calcd. for [(M+Na)⁺]=641.4397, found=641.4383.

### R)-5-((1R,3aS,7aR,E)-4-(2-((3R,5R)-3,5-dihydroxycyclohexylidene)ethylidene)-7a-methyl-octahydro-1H-inden-1-yl)-N-hydroxyhexanamide (36)

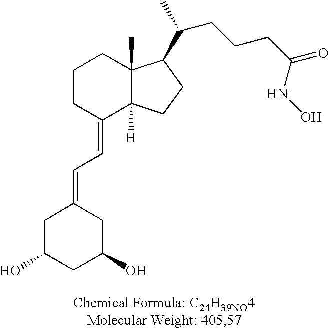

In a flame dried round bottom flask under argon, a solution of acid 35 (63.0 mg, 0.102 mmol) in dry CH₂Cl₂ (2 mL) and dry DMF (1 μL) was cooled to 0° C. followed by the addition of oxalylchloride (12.9 μL, 0.153 mmol). The solution was left to stir at 0° C. for 90 min. A solution of DIPEA (53.3 μL, 0.306) and TBSONH₂ (30.0 mg, 0.204 mmol) in CH₂Cl₂ (1 mL) was then added. The reaction mixture was left to stir at 0° C. for 1 h and then at rt. for an additional 1 h. A solution of citric acid (10 mL, 1 M) was then added, the mixture extracted with EtOAc (3×10 mL) and the combined organic layer washed with H₂O (10 mL), brine (10 mL) and dried over MgSO₄. The solution was concentrated in vacuo and the crude material dissolved in CH₂Cl₂ (1 mL) and CH₃CN (1 mL). HF (70 μL of a 48% solution in H₂O) was added and the solution stirred at rt. for 2 h. The reaction was quenched with caution by the addition of a saturated solution of NaHCO₃ until no further effervescence was observed. The reaction mixture was extracted with CH₂Cl₂ (3×5 mL) and the combined organic layers washed with H₂O (5 mL), brine (5 mL), dried over MgSO₄ and then concentrated in vacuo. The crude product was purified by means of octadecyl-functionalized silica gel column chromatography using a solvent gradient starting from distilled water and ending with pure methanol to afford 11 mg (0.03 mmol) of hydroxamic acid 36 in 27% yield. R_{f}=0.30 [(88:10:2) CH₂Cl₂:MeOH:CH₃COOH]; ¹H NMR (400 MHz, CD₃OD) δ 6.22 (1H, d, J=11.0 Hz), 5.89 (1H, d, J=11.0 Hz), 4.08-3.92 (2H, m), 2.83 (1H, br d, J=12.4 Hz), 2.59 (1H, br d, J=13.2 Hz), 2.52-1.04 (27H, m), 0.97 (3H, d, J=3.2 Hz), 0.58 (3H, s); ¹³C NMR (400 MHz, CD₃OD) δ 176.6, 140.9, 132.7, 122.3, 116.0, 66.8, 66.5, 56.6, 56.3, 45.6, 44.2, 41.5, 40.7, 36.5, 36.1, 35.3, 34.2, 28.7, 27.5, 23.4, 22.1, 21.6, 18.1, 11.3; IR (KBr) ν 3390 (br), 2941, 2870, 1709, 1439, 1376, 1212, 1046 cm⁻¹; HRMS (ESI): m/z calcd. for [(M+Na)⁺]=428.2777, found=428.2781.

### (6S)-6-((1R,3R,7E,17β)-1,3-bis[tert-butyl(dimethyl) silyl-oxy]-9,10-secoestra-5,7-dien-17-yl)heptanal (37)

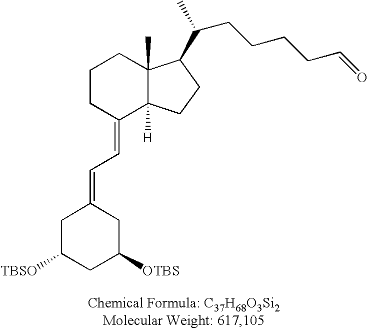

In a flame dried round bottom flask under argon atmosphere, a 1 M solution of NaHMDS in THF (1.0 mL, 1.0 mmol, 10 equiv) was added to a solution of methoxymethyltriphenylphosphonium chloride (360 mg, 1.0 mmol, 10 equiv) in dry THF (10 mL). The reaction vessel was suspended above the ice bath for 30 min, then recooled to −78° C. To this solution was added via cannula over a period of 5 min a solution of 34 (60 mg, 0.100 mmol, 1 equiv) in dry THF (1 mL). The reaction was left to stir at −78° C. for 1 h then warmed to 0° C. for 4 h, and quenched with sat. NH₄Cl (25 mL). The layers were separated and the aqueous layer extracted with EtOAc (2×25 mL). The organic layers were combined and extracted with sat. NH₄Cl (2×25 mL), H₂O (25 mL) and brine (25 mL), then dried (MgSO₄), and concentrated in vacuo then purified by silica gel column chromatography (5% EtOAc in hexanes) to obtain 40 mg of a mixture 7/3 of E/Z enol ethers in 63% yield. R_{f}=0.6 (5% EtOAc in hexanes); ¹H NMR (400 MHz, CDCl₃) δ6.28 (0.6H, d, J=12.8 Hz), 6.17 (1H, d, J=11.0 Hz), 5.86 (0.3H, d, J=6.4 Hz), 5.81 (1H, d, J=11.0 Hz), 4.73 (0.7H, dt, J=12.4, 7.2 Hz), 4.33 (0.3H, q, J=6.8), 4.12-4.01 (2H, m), 3.58 (0.9H, s), 3.50 (2.1H, s), 2.81 (1H, d, J=12.0 Hz), 2.43-2.33 (2H, m), 2.25 (1H, d, J=13.6 Hz), 2.15-1.72 (6H, m), 1.70-1.17 (14H, m), 1.10-0.98 (1H, m), 0.91 (3H, d, J=6.4), 8.87 (9H, s), 8.86 (9H, s), 0.53 (3H, s), 0.05 (12H, s). Enol ether (40 mg, 0.063 mmol) was dissolved in a solution of CHCl₃ (1 mL), distilled H₂O (0.5 mL) and TFA (0.15 mL), and cooled to 0° C. The reaction was stirred at 0° C. for approx. 30 min and monitored by TLC. Until complete consumption of the starting material, the reaction was quenched with sat. NaHCO₃ (5 mL). CH₂Cl₂ (10 mL) was added to the mixture, the layers were separated and the aqueous layer extracted with CH₂Cl₂ (2×10 mL). The organic layers were combined and washed with sat. NaHCO₃ (2×25 mL), distilled H₂O (25 mL) and brine (25 mL), then dried (MgSO₄), and concentrated in vacuo to give the crude product. 37 was isolated via FCC (10% EtOAc in hexanes) as a clear oil in 94% yield (36.5 mg, 0.059 mmol). R_{f}=0.2 (10% EtOAc in hexanes); ¹H NMR (400 MHz, CDCl₃) δ9.77 (1H, t, J=1.6 Hz), 6.16 (1H, d, J=10.0 Hz), 5.81 (1H, d, J=10.0 Hz), 4.12-4.12-4.02 (2H, m), 2.81 (1H, d, J=11.6 Hz), 2.43 (2H, t, J=7.2 Hz), 2.37 (2H, dd, J=12.4, 5.2 Hz), 2.25 (1H, d, J=11.6 Hz), 2.10 (1H, dd, J=12.6, 8.2 Hz), 2.04-1.93 (2H, m), 1.93-1.75 (2H, m), 1.70-1.45 (8H, m), 1.45-1.15 (7H, m), 1.15-1.00 (1H, m), 0.91 (3H, d, J=6.0 Hz), 0.87 (9H, s), 0.86 (9H, s), 0.53 (3H, s), 0.05 (12H, s); ¹³C NMR (400 MHz, CDCl₃) δ 203.3, 141.0, 133.9, 121.9, 116.4, 68.3, 68.2, 56.6, 56.5, 46.3, 45.9, 44.3, 43.9, 40.8, 37.0, 36.2, 35.8, 28.9, 27.9, 26.11, 26.08, 25.9, 23.6, 22.8, 22.5, 19.0, 18.4, 18.3, 12.3, −4.4, −4.5, −4.6, −4.7; IR (KBr) ν 2949, 2884, 2856, 1728, 1468, 1252, 1052, 1025, 960, 920, 837, 776 cm⁻¹; HRMS (ESI): m/z calcd. for [(M+Na)⁺]=639.4605, found=639.4599.

(6S)-6-((1R,3R,7E,17β)-1,3-bis[tert-butyl(dimethyl)silyl-oxy]-9,10-secoestra-5,7-dien-17-yl)heptanoic acid (38) was prepared following the procedure as for 35.

(R)-5-((1R,3aS,7aR,E)-4-(2-((3R,5R)-3,5-dihydroxy cyclohexyl idene)ethylidene)-7a-methyl-octahydro-1H-inden-1-yl)-N-hydroxyheptanamide (39) was prepared from 38 following the same procedure as for 36.

It is to be understood that the invention is not limited in its application to the details of construction and parts as described hereinabove. The invention is capable of other embodiments and of being practiced in various ways. It is also understood that the phraseology or terminology used herein is for the purpose of description and not limitation. Hence, although the present invention has been described hereinabove by way of illustrative embodiments thereof, it can be modified, without departing from the spirit, scope and nature of the subject invention as defined in the appended claims.

### REFERENCES

1. Vitamin D; Feldman, D.; Glorieux, F. H.; Pike, J. W. Eds.; Academic Press: New York, 1997.
2. Lin, R.; White, J. H. BioEssays, 2004, 26, 21-28.
3. Ordonez-Moran, P.; Larriba, M. J.; Pendas-Franco, N.; Aguilara, O.; Gonzalez-Sanco, J. M.; Munoz, A. Front. Biosci. 2005, 10, 2723-2749 Suppl. S.
4. Bouillon, R.; Okamura, W. H.; Norman, A. W. Endocrinol. Rev. 1995, 16, 200-257.
5. Nagpal, S.; Lu, J.; Boehm, M. F. Curr. Med. Chem. 2001, 8, 1679-1697.
6. Guyton, K. Z.; Kensler, T. W.; Posner, G. H. Annu. Rev. Pharmacol. Toxicol. 2001, 41, 421-442.
7. Calcipotriol for treatment of psoriasis and 1α,25-dihydroxy-19-nor-vitamin D₂ for treatment of secondary hyperparathyroidism, see: Holick, M. F. Vitamin D; Humana: Totowa, 1999.
8. McLaughlin, F.; La Thangue, N. B. Biochem. Pharm. 2004, 68, 1139-1144.
9. Villar-Garea, A.; Esteller, M. Int. J. Cancer 2004, 112, 171-178.
10. Rashid, S. F; Moore, J. S; Walker E.; Driver, P. M.; Engel, J.; Edwards, C. E.; Brown, G.; Uskokovic, M. R.; Campbell, M. J. Oncogene 2001; 20: 1860-72.
11. Buerger, C; Groner, B. J. Cancer Res. Clin. Oncol. 2003, 129, 669-75.
12. Luis Abad, J.; Gonzalez, M. A.; del Real, G.; Mira, E.; Manes, S.; Serrano, F.; Bernad, A. Mol. Ther. 2003, 8, 475-84.
13. Bralet, J.; Schwartz, J.-C. Trends in Pharmacolog. Sci. 2001, 22, 106-09.
14. Rochel, N.; Wurtz, J. M.; Mitschler, A.; Klaholz, B.; Moras, D. Mol. Cell. 2000, 5, 173-179.
15. Fujishima, T.; Konno, K.; Nakagawa, K.; Kurobe, M.; Okano, T.; Takayama, H. Biorg. Med. Chem. Lett. 2000, 8, 123-134.
16. Verstuyf, A.; Verlinden, L.; Van Etten, E.; Shi, L.; Wo, Y.; D'Halleweyn, C.; Van Haver, D.; Zhu, G.-D.; Chen Y.-J.; Zhou, X.; Haussler, M. R.; De Clercq, P.; Vandewalle, M.; Van Baelen, H.; Mathieu, C.; Bouillon, R. J. Bone Min. Res. 2000, 15, 237-252.
17. Hilpert, H.; Wirz, B. Tetrahedron 2001, 57, 681-684.
18. Perlman, K. L.; Sicinski R. R.; Schnoes, H. K.; DeLuca H. F. Tetrahedron Lett. 1990, 31, 1823-1824.
19. Finnin, M. S.; Donigian, J. R.; Cohen, A.; Richon V. M.; Rifkind R. A.; Marks, P. A.; Breslow, R.; Pavletich, N. P. Nature 1991, 401, 188-193.
20. Marson, C. M.; Serradji, N.; Rioja, A. S.; Gastaud, S. P.; Alao, J. P.; Coombes, R. C.; Vigushin, D. M. Bioorg. Med. Chem. Lett. 2004, 14, 2477-2481.
21. Perlman, K. L.; Swenson, R. E.; Paaren, H. E.; Schnoes, H. K.; DeLuca, H. F. Tetrahedron Lett. 1991, 32, 7663-7665.
22. Graham, A. E.; Thomas, A. V.; Yang. R. J. Org. Chem. 2000, 65, 2583.
23. Chou S. C; Azuma, Y.; Varia, M. A.; Raleigh, J. A. British J. Cancer 2004, 90, 728-35.
24. Akutsu, N.; Lin, R.; Bastien, Y.; Bestawros, A.; Enepekides, D. J.; Black, M. J.; White, J. H. Mol. Endocrinol. 2001, 15, 1127-39.
25. Ferrara, J.; McCuaig, K.; Hendy, G. N.; Uskokovic, M.; White, J. H. J. Biol. Chem., 1994, 269, 2971-81.
26. Tavera-Mendoza, L.; Wang, T. T.; Lallemant, B.; Zhang, R.; Nagai, Y.; Bourdeau, V.; Ramiro Calderon, M.; Desbarats, J.; Mader, S.; White, J. H. EMBO Rep. 2006, 7, 180-185.
27. Akutsu, N., Lin, R., Bastien, Y., Bestawros, A., Enepekides, D. J., Black, M. J. and White, J. H. Mol. Endocrinol., 2001, 15, 1127-39.
28. Moore, P. S., Barbi, S., Donadelli, M., Costanzo, C., Bassi, C., Palmieri, M., and Scarpa, A. Biochim. Biophys. Acta., 2004, 1693, 167-176.
29. Hubbert, C., Guardiola, A., Shao, R., Kawaguchi, Y., Ito, A., Nixon, A., Yoshida, M., Wang, X.-F., and Yao, T.-P. Nature, 2002, 417, 455-58.
30. Dowling, M., Voong, K. R., Kim, M., Keutmann, M. K., Harris, E., and Kao, G. D. Cancer Biol.& Ther. 2005, 4, 197-206.
31. Cho, H. P., Liu, Y., Gomez, M., Dunlap, J., Tyers, M., and Wang Y. Mol. Cell. Biol., 2005, 25, 4541-4551.
32. Inhoffen, H. H.; Quinkert, G.; Schutz, S.; Friedrich, G.; Tober, E. Chem. Ber. 1958, 91, 781.
33. Mascareñ as, J. L.; Mouinõ, A.; Castedo, L.; J. Org. Chem. 1986, 51, 1269-1272.

What is claimed is:

1. A hybrid molecule comprising a vitamin D receptor agonist moiety and an HDAC inhibitor moiety.

2. The hybrid molecule of claim 1, wherein the HDAC inhibitor moiety is modelled after an HDAC inhibitor selected from the group consisting of TSA, sodium butyrate (NaB), valproic acid, N-acetyldinaline, and suberoylanilide hydroxamic acid (SAHA).

3. The hybrid molecule of claim 1, wherein the HDAC inhibitor moiety is derived from TSA.

4. The hybrid molecule of claim 1, wherein the HDAC inhibitor moiety is derived from SAHA.

5. The hybrid molecule of claims 3 and 4 or a pharmaceutically acceptable salt or prodrug thereof, as represented by a structure selected from the group consisting of: 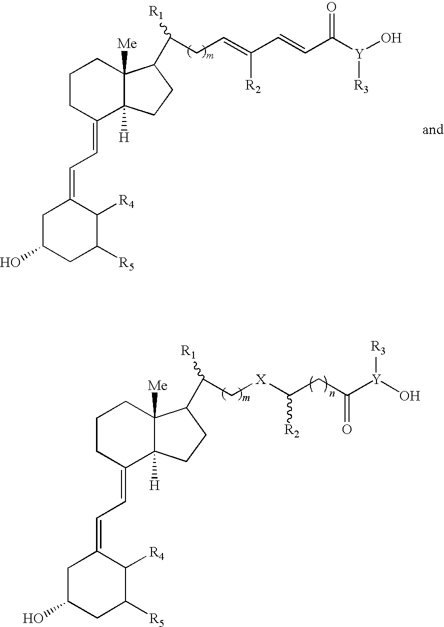
wherein:
R₁, R₂, R₃, and R₄ are independently selected from the group consisting of H, lower alkyl, and alkylene;
R₅ is selected from the group consisting of H and OH;
X is selected from the group consisting of O, S NH and CH₂;
Y is selected from the group consisting of N and CH;
m is an integer ranging from 0 to 3; and
n is an integer ranging from 1 to 3.

6. The hybrid molecule of claim 5, or a pharmaceutically acceptable salt or prodrug thereof comprising the structure:

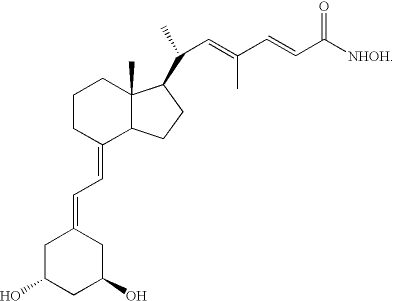

7. The hybrid molecule of claim 5, or a pharmaceutically acceptable salt or prodrug thereof comprising the structure:

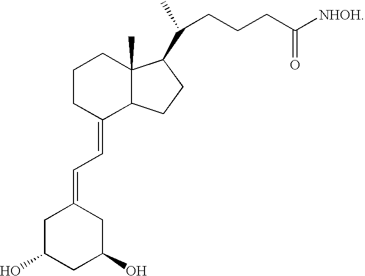

8. The hybrid molecule of claim 5, or a pharmaceutically acceptable salt or prodrug thereof comprising the structure:

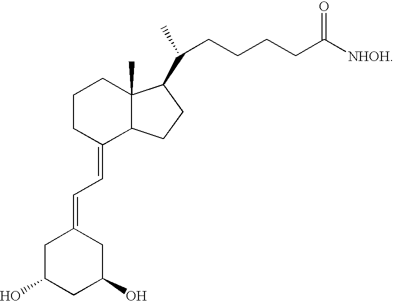

9. A method for the treatment of disorders or diseases wherein inhibition of HDAC and/or vitamin D agonism is beneficial, said method comprising administering to a subject in need thereof and affective amount of one or more hybrid molecules of claim 1.

10. A method of treating a patient afflicted with a condition selected from the group consisting of cancer, inflammation and auto-immune diseases, comprising administering to the patient a therapeutically effective amount of one or more hybrid molecules of claim 1.

11. A method of inducing wound healing comprising, administering to a patient in need thereof a therapeutically effective amount of one or more hybrid molecules of claim 1.

12. A method of treating bacterial infections in a patient comprising, administering to the patient a therapeutically effective amount of one or more hybrid molecules of claim 1.

13. A method of reducing proliferation of/or inducing cell death in neoplastic cells comprising, contacting said neoplastic cells with one or more of the hybrid molecules of claim 1.

14. Use of one or more of the hybrid molecules of claim 1 in the manufacture of a medicament for the treatment of a condition selected from the group consisting of cancer, inflammation and auto-immune diseases.

15. Use of one or more of the hybrid molecules of claim 1 in the manufacture of a medicament for inducing wound healing.

16. Use of one or more of the hybrid molecules of claim 1 in the manufacture of a medicament for treating bacterial infections.

17. A pharmaceutical composition comprising an effective amount of one or more of the hybrid molecules of 1 in association with one or more pharmaceutically acceptable carriers, excipients or diluents.

18. An admixture comprising an effective amount of one or more of the hybrid molecules of claim 1 in association with one or more pharmaceutically acceptable carriers, excipients or diluents.
